SEQUENCE LISTING (22 sequences)

SEQ ID NO: 1 - DNA, 21 bp, Artificial
  Designed oligonucleotide based on Mycobacterium tuberculosis rpoBgene
agtgcaaagacaaggacatga

SEQ ID NO: 2 - DNA, 20 bp, Artificial
  Designed oligonucleotide based on Mycobacterium tuberculosis rpoBgene
ttctcggtcatcatcgggaa

SEQ ID NO: 3 - DNA, 18 bp, Artificial
  Designed oligonucleotide based on Mycobacterium tuberculosis rpoBgene
gatgtcgttgtcgttctc

SEQ ID NO: 4 - DNA, 18 bp, Artificial
  Designed oligonucleotide based on Mycobacterium tuberculosis rpoBgene
accgtctgactcttgatc

SEQ ID NO: 5 - DNA, 35 bp, Artificial
  Designed oligonucleotide based on Mycobacterium tuberculosis rpoBgene
cgcgatgtcaccgccgagttcatcaacaaatcgcg

SEQ ID NO: 6 - DNA, 24 bp, Artificial
  Designed oligonucleotide based on Lambda DNA
acctcattttcatgtccggtcagc

SEQ ID NO: 7 - DNA, 24 bp, Artificial
  Designed oligonucleotide based on Lambda DNA
ggcagagctgaaagaggagcttga

SEQ ID NO: 8 - DNA, 28 bp, Artificial
  Designed oligonucleotide based on Mycobacterium tuberculosis rpoBgene
ccatgaacaccgtctgactcttgatctc

SEQ ID NO: 9 - DNA, 26 bp, Artificial
  Designed oligonucleotide based on Mycobacterium tuberculosis rpoBgene
ccatgaacaccgtctgactcttgatc

SEQ ID NO: 10 - DNA, 24 bp, Artificial
  Designed oligonucleotide based on Mycobacterium tuberculosis rpoBgene
ccatgaacaccgtctgactcttga

SEQ ID NO: 11 - DNA, 22 bp, Artificial
  Designed oligonucleotide based on Mycobacterium tuberculosis rpoBgene
ccatgaacaccgtctgactctt

SEQ ID NO: 12 - DNA, 20 bp, Artificial
  Designed oligonucleotide based on Mycobacterium tuberculosis rpoBgene
ccatgaacaccgtctgactc

SEQ ID NO: 13 - DNA, 27 bp, Artificial
  Designed oligonucleotide based on Mycobacterium tuberculosis rpoBgene
ccatgaacaccgtctgaccttgatctc

SEQ ID NO: 14 - DNA, 25 bp, Artificial
  Designed oligonucleotide based on Mycobacterium tuberculosis rpoBgene
ccatgaacaccgtctgctcttgatc

SEQ ID NO: 15 - DNA, 23 bp, Artificial
  Designed oligonucleotide based on Mycobacterium tuberculosis rpoBgene
ccatgaacaccgtcgactcttga

SEQ ID NO: 16 - DNA, 21 bp, Artificial
  Designed oligonucleotide based on Mycobacterium tuberculosis rpoBgene
ccatgaacaccgctgactctt

SEQ ID NO: 17 - DNA, 19 bp, Artificial
  Designed oligonucleotide based on Mycobacterium tuberculosis rpoBgene
ccatgaacacgtctgactc

SEQ ID NO: 18 - DNA, 43 bp, Artificial
  Designed oligonucleotide based on Mycobacterium tuberculosis rpoBgene
cccttcagtgggtacttgtggcagactgagaactagagtggcc

SEQ ID NO: 19 - DNA, 20 bp, Artificial
  Designed oligonucleotide based on Mycobacterium tuberculosis rpoBgene
caagagtcagacggtgttca

SEQ ID NO: 20 - DNA, 20 bp, Artificial
  Designed oligonucleotide based on Mycobacterium tuberculosis rpoBgene
ttgtcggtggacttgtcaat

SEQ ID NO: 21 - DNA, 22 bp, Artificial
  Designed oligonucleotide based on Mycobacterium tuberculosis rpoBgene
tgacttcccgatgatgacctag

SEQ ID NO: 22 - DNA, 22 bp, Artificial
  designed oligonucleotide based on Mycobacterium tuberculosis rpoBgene
tgacttcccgatgatgaccgag

(19) United States
(12) Patent Application Publication
(10) Pub. No.: US 2008/0220415 A1
(43) Pub. Date: Sep. 11, 2008

(54) Detection Method of Dna Amplification Using Probe Labeled With Intercalating Dye

(76) Inventors: Han Oh Park, Deajeon (KR); Hyun-Bae Kim, Daejeon (KR); Sung-Min Chi, Chungcheongnam-do (KR)

Correspondence Address: NATH & ASSOCIATES, 112 South West Street, Alexandria, VA, 22314, US

(21) Appl. No.: 10/593,900

(22) PCT Filed: Mar. 25, 2005
(86) PCT No.: PCT/KR05/00889

§ 371 Date: Sep. 22, 2006

(30) Foreign Application Priority Data
Mar. 25, 2004 (KR) 10-2004-0020444

(51) Int. Cl. C12Q 1/68

(52) U.S. Cl. 435/6

(57) ABSTRACT

The present invention relates to a detection method of nucleic acid amplification using probe labeled with intercalating dye. More particularly, the present invention is directed to a real-time detection method of nucleic acid amplification, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said doublestranded nucleic acid into single strands by heating the aqueous buffer prepared in step i) up to 931 C to 96 C; iii) annealing said pair of primers with said single strand by cooling the solution obtained in step ii) up to 50 C. to 571 C; iv) replicating said single-stranded nucleic acid by heating the solution obtained from step iii) up to 701 C to 74° C.; v) denaturing said replicated nucleic add into single strands by heating the solution obtained in step iv) up to 931 C to 961 C; vi) annealing said fluorescent probe with said single-stranded nucleic acid by cooling the solution obtained in step v up to 501 C to 57 C; vii) measuring an intensity of the fluorescence emitted from the solution obtained in step vi); and viii) repeating more than one steps iv) through vii).

### TECHNICAL FIELD

The present invention relates to a detection method of nucleic acid amplification using probe labeled with intercalating dye. More particularly, the present invention is directed to a real-time detection method of nucleic acid amplification, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said double-stranded nucleic acid into single strands by heating the aqueous buffer prepared in step i) up to 93° C. to 96° C.; iii) annealing said pair of primers with said single strand by cooling the solution obtained in step ii) up to 50° C. to 57° C.; iv) replicating said single-stranded nucleic acid by heating the solution obtained from step iii) up to 70° C. to 74° C.; v) denaturing said replicated nucleic acid into single strands by heating the solution obtained in step iv) up to 93° C. to 96° C.; vi) annealing said fluorescent probe with said single-stranded nucleic acid by cooling the solution obtained in step v) up to 50° C. to 57° C.; vii) measuring an intensity of the fluorescence emitted from the solution obtained in step vi); and viii) repeating more than one steps iv) through vi).

### BACKGROUND ART

The conventional polymerase chain reaction (PCR) can provide some information related to only the size of an amplified part through the analysis of PCR products separated from an agarose gel. The information on the base sequence of an amplified part, can only be presumed.

When a specificity of a PCR product band separated in agarose gel is suspected, the base sequence of the corresponding band, should be analyzed, or a southern blot analysis should be carried out by using a probe labeled with isotope.

However, the experiment for the confirmation of the specificity of the suspeted band takes long time more than two or three days.

Moreover, the stringency required for the annealing of a probe, should be controlled under various conditions.

Therefore, in order to overcome the demerit of the conventional end-point detection, the real-time PCR technique wherein which a concentration of a nucleic acid which exists in a sample can be calculated exactly by analyzing the signals obtained from each cycles of PCR has been developed.

Now, accurate, prompt and sensitive results can be obtained through the real-time PCR technique which employs a special probe.

The real-time PCR which adds a quantitative analysis function on the conventional PCR method, is a new method which can automate several routine procedures and can provide accurate results by reducing possible errors.

In this real-time PCR technique, a fluorescent reporter dye is employed to monitor all procedures of entire reactions.

The intensity of fluorescence is emitted from the sample amplified, is increased in proportion to an amount of amplified products accumulated through each cycles of PCR. Even though a detector cannot detect fluorescence at an early stage of PCR, an accumulated fluorescence can be detected as the amplified products are accumulated.

A threshold cycle(C (T)) is an amplification frequency at the point of time when fluorescence intensity is detectable.

A proportional relationship is established between a log value of an initial amount of nucleic acids and the threshold cycle.

Therefore, a standard calibration curve can be established by measuring a threshold cycle of a sample in which an initial amount of nucleic acid is known. And an initial amount of a nucleic acid in a sample, in which an initial amount of a nucleic acid is unknown, can be correctly calculated by referring said standard calibration curve.

Meanwhile, methods which can calculate a correct concentration of nucleic acids in a sample by analyzing samples obtained from each cycles of real-time PCR, are such as a method which employs the Taqman probe (Holland et al., 1991, Proc. Natl. Acad. Sci. USA 88:7276-7280; Lee et al., 1993, Nucleic Acids Res. 21:3761-3776), a method which uses the Molecular Beacon probe (Tyagi & Kramer 1996, Nature Biotech. 14:303-309, U.S. Pat. No. 5,119,801, U.S. Pat. No. 5,312,728), a method which employs an intercalating agent such as SYBR Green I and a method which uses the adjacent hybridization probe (U.S. Pat. No. 5,210,015).

The TaqMan probe is a probe wherein a reporter dye (6-FAM, JOE, VIC, HEX, TET, Fluorescein, Cy-dyes) is attached at the 5′ end and a quencher (TAMRA) is attached at the 3′ end.

The probe cannot be operated as a primer because the 3′ end of a probe is blocked so that DNA synthesis cannot begin from the 3′ end of a probe. Native Taq enzyme has a 5′ nuclease activity, which has a function to remove downstream DNA hybridized with template DNA.

The 5′ nuclease function of Taq enzyme can be activated when the 5′ end of downstream DNA is double-strand and Taq enzyme is bonded at the 3-OH of upstream DNA (Livak, K. J., J. Marmaro, and S. Flood. 1995. Guidelines for designing Taqman fluorescent probes for 5′ nuclease assays, Research news. PE Applied Biosystems, Foster city, Calif.).

When DNA replication is started, the 5′ end of the probe is removed by the 5′ nuclease activity of Taq enzyme.

Fluorescence Intensity (FI) of the TaqMan probe is inversely proportional to multiplication of the distance (R) between the reporter dye and the quencher by 6. Before the 5′ end of the probe is removed by Taq enzyme, a signal of the reporter dye is disrupted by fluorescence resonance energy transfer (FRET). However, after the 5′ end of the probe is removed and the quencher dye is extricated from the 5′ end of the probe, the reporter dye emits light.

When one molecule of target DNA is synthesized, the 5′ end of one molecule of the probe is removed and the reporter dye is separated with the quencher dye (U.S. Pat. No. 5,763,181, U.S. Pat. No. 5,691,146 etc). Therefore, a signal of the reporter dye is increased in proportion to the amount of amplified DNA.

Even though the information for the size of amplified DNA, cannot be obtained by using the TaqMan method, information of a base sequence and an amount of amplified DNA, can be known by using the TaqMan probe which has a very high specificity.

However, the TaqMan probe method cannot-perfectly quench the reporter dye since the probe is composed of at least 20 bases. Moreover, the production of the TaqMan probe is difficult and expensive.

A method which uses the Molecular Beacon probe, is employed to analyze specific DNA and detect RNA in living cell. The Molecular Beacon probe has a hair pin shaped structure. The roof part of the Molecular Beacon probe is a single-stranded nucleic acid which is complementary to at least a part of a target nucleic acid. And a stem part of the Molecular Beacon probe is formed by annealing two complementary arm sequences at both ends of the probe (Tyagi, S. and F, R, Kramer. 1996, Molecular beacons that fluoresce upon hybridization, Nat., Biotechnol., 16:49).

A reporter dye of the 5′ end of the Molecular Beacon probe, is selected from the group which consists of Texas Red, Rodamin Red, FAM, HEX, TET, ROX, TAMRA, Fluorescein or Oregon green, etc., and quencher of the 3′ end of the Molecular Beacon probe is DABSYL [4-(4′-dimethylaminophenylazo)benzoic acid].

The Molecular Beacon probe cannot emit fluorescence when the probe is not hybridized with a target nucleic acid. Therefore, detection procedure for the probe, can be simplified.

When a reporter dye and a quencher dye of a stem part are close to each other, fluorescence of the reporter dye is quenched. However, when a target nucleic acid is contacted, more longer and stable hybridization with the target nucleic acid is formed instead of hybridization with its own arm sequence. Therefore, the reporter dye is not close to the quencher and fluorescence can be detected.

However, the method which employs the Molecular Beacon probe is inconvenient for separating the probe from the target nucleic acid (Matsuo, T. 1998. In situ visualization of messenger RNA for basic fibroblast growth factor in livingcells. Biochim. Biophys. Acta 1379:178-184). Further, the production of the Molecular Beacon probe is difficult and expensive since the probe is composed of at least 30 to 40 bases.

Another analysis method is a method, which uses the adjacent hybridization probe. This method employs two probes designed to hybridize with a target nucleic acid as head-to-tail arrangement.

In the method, the 3′ end of one probe is bonded with a fluorescence donor and the 5′ end of another probe is bonded with a fluorescence acceptor. Therefore, when the fluorescence donor and fluorescence acceptor are adjacent, fluorescence energy transfer is occurred and emission light of the donor functions as excitation light of the accepter.

Energy coming from a donor excited by a light resource, is transferred to an acceptor and the acceptor emits fluorescence. The fluorescence intensity, is proportional to an amount of a probe hybridized with amplified nucleic acids. The method is employed to detect DNA and single base modification of a nucleic acid.

However, the processes of this method, are complicated since the method needs 4 kinds of primers. In addition, the specificity of this method, is in a low level (Heller, M. J. and L. E. Morrison. 1985. Chemiluminescent and fluorescence probes for DNA hybridization, p. 245-256. In D. T. Kinsbury and S. Flakow (Eds.), Rapid Detection and Identification of Infectious Agents, Academic Press, New York).

Still another analysis method is a method which uses a intercalating dye such as SYBR Green I, Foerst 33228, Etidium Bromide (EtBr), etc.

The method can be applied to wide range of samples on the ground that intercalating dye of the method can be bonded to double-stranded DNA without special target sequences.

In a method that uses an intercalating dye, processes are simple because it is easy to produce a probe and to separate a probe from a nucleic acid. Also, because control of processes is easy, it is convenient to obtain a signal from a particular step.

However, the method has a demerit that amplified products of primers, non-specific amplified products, etc. are easily produced. Because of said background noises, it is needed to measure a melting temperature of amplified products and confirm products obtained from PCR by analyzing a melting curve (Higuchi, R., Dollinger, G., Walsh, P. S., and Griffith, R. 1992. Simultaneous amplification and detection of specific DNA sequences, Biotechnology 10:413, Higuchi, R., Fockler, C., Dollinger, G., and Watson, R. 1993, kinetic PCR: Real monitoring of DNA amplification reactions. Biotechnology 11:1026).

In order to obviate above mentioned problems, the present inventors have tried to exploit a novel probe and a detection method-of amplified nucleic acid which can promptly perform a correct analysis of amplified nucleic acid, while production of a probe and analysis procedure are easy and convenient.

### DISCLOSURE

### [Technical Solution]

The primary object of the present invention is to provide a fluorescent probe for real-time detection of amplification of nucleic acid, wherein a fluorescent dye of which intensity of fluorescence is varied when the dye is intercalated into a double-stranded nucleic acid, is connected with an oligonucleotide of which base sequence is complementary with at least a part of said nucleic acid.

Another object of the present invention is to provide a real-time detection method of nucleic acid amplification, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said double-stranded nucleic acid into single strands by heating the aqueous buffer prepared in step i) up to 93° C. to 96° C.; iii) annealing said pair of primers with said single strand by cooling the solution obtained in step ii) up to 50° C. to 57° C.; iv) replicating said single-stranded nucleic acid by heating the solution obtained from step iii) up to 70° C. to 74° C.; v) denaturing said replicated nucleic acid into single strands by heating the solution obtained in step iv) up to 93° C. to 96° C.; vi) annealing said fluorescent probe with said single-stranded nucleic acid by cooling the solution obtained in step v) up to 50° C. to 57° C.; vii) measuring an intensity of the fluorescence emitted from the solution obtained in step vi); and viii) repeating more than one steps iv) through vii).

Further object of the present invention is to provide a real-time detection method of initial amount of a nucleic acid in a sample, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said double-stranded nucleic acid into single strands by heating the aqueous buffer prepared in step i) up to 93° C. to 96° C.; iii) annealing said pair of primers with said single strand by cooling the solution obtained in step ii) up to 50° C. to 57° C.; iv) replicating said single-stranded nucleic acid by heating the solution obtained from step iii) up to 70° C. to 74° C.; v) denaturing said replicated nucleic acid into single strands by heating the solution obtained in step iv) up to 93° C. to 96° C.; vi) annealing said fluorescent probe with said single-stranded nucleic acid by cooling the solution obtained in step v) up to 50° C. to 57° C.; vii) measuring an intensity of the fluorescence emitted from the solution obtained in step vi); viii) repeating more than one steps iv) through vii); ix) establishing a standard calibration curve which indicates the correlation between the log value of an initial amount of the nucleic acid and a threshold cycle shown by the performance of above steps i) through viii), by using a sample of which an initial amount of the nucleic acid is known; and x) detecting an initial amount of the nucleic acid based on the log value which corresponds to the threshold cycle obtained from the performance of said steps i) through vi), by referring to the standard calibration curve obtained in step ix).

Still another object of the present invention is to provide a composition for the amplification of a nucleic acid which comprises i) a pair of primers for amplification of said nucleic acid; ii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iii) DNA polymerase; and iv) four (4) kinds of nucleotides.

Yet another object of the present is to provide a real-time detection method of the nucleic acid amplification, comprising the steps of i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase; ii) replicating a single-stranded cDNA by heating the aqueous buffer prepared in step i) up to 42° C. to 50° C.; iii) denaturing a primer for a reverse transcription and a reverse transcriptase from said single-stranded cDNA by heating the solution obtained from said step ii) up to 93° C. to 96° C.; iv) annealing the pair of primers with said single-stranded nucleic acid by cooling the solution obtained from said step iii) up to 50° C. to 57° C.; v) replicating said single-stranded nucleic acid by heating the solution obtained from step iv) up to 70° C. to 74° C.; vi) denaturing said replicated nucleic acid into single strands by heating the solution obtained from step v) up to 93° C. to 96° C.; vii) annealing said fluorescent probe with said single-strand nucleic acid by cooling the solution obtained from step vi) up to 50-57° C.; viii) measuring an intensity of the fluorescence emitted from the solution obtained in step vii); and ix) repeating more than one steps v) through viii).

Yet another object of the present invention is to provide a real-time detection method of initial amount of a nucleic acid in a sample, comprising the steps of i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase; ii) replicating a single-stranded cDNA by heating the aqueous buffer prepared in step i ) up to 42° C. to 50° C.; iii) denaturing a primer for a reverse transcription and a reverse transcriptase from said single-stranded cDNA by heating the solution obtained from said step ii) up to 93° C. to 96° C.; iv) annealing the pair of primers with said single-stranded nucleic acid by cooling the solution obtained from said step iii) up to 50° C. to 57° C.; v) replicating said single-stranded nucleic acid by heating the solution obtained from step iv) up to 70° C. to 74° C.; vi) denaturing said replicated nucleic acid into single strands by heating the solution obtained from step v) up to 93° C. to 96° C.; vii) annealing said fluorescent probe with said single-strand nucleic acid by cooling the solution obtained from step vi) up to 50-57° C.; viii) measuring an intensity of the fluorescence emitted from the solution obtained in step vii); ix) repeating more than one steps v) through viii); x) establishing a standard calibration curve which indicates the correlation between the log value of an initial amount of the nucleic acid and a threshold cycle shown by the performance of above steps i) through ix), by using a sample of which an initial amount of the nucleic acid is known; and xi) detecting an initial amount of the nucleic acid based on the log value which corresponds to the threshold cycle obtained from the performance of said steps i) through ix), by referring to the standard calibration curve obtained in step ix).

Yet another object of the present invention is to provide a composition for the amplification of a nucleic acid which comprises i) a pair of primers for amplification of said nucleic acid; ii) a primer for reverse transcription iii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iv) DNA polymerase; v) a reverse transcriptase and iv) four (4) kinds of nucleotides.

The above mentioned object of the present invention can be achieved by providing a fluorescent probe for real-time detection of amplification of nucleic acid, wherein a fluorescent dye of which intensity of fluorescence is varied when the dye is intercalated into a double-stranded nucleic acid, is connected with an oligonucleotide of which base sequence is complementary with at least a part of said nucleic acid.

The fluorescent probe is blocked so that replication cannot begin from said 3′ end and is composed of 10 to 40 bases.

Preferably, the probe comprises the base sequence selected from the group which consists of the Seq. ID Nos. 1 through 22.

Also, an intercalating dye of the probe is operated as a fluorescent dye and an intercalating dye is desirable to be selected from a group which consists of Acridine homodimer and these derivatives, Acridine Orange and these derivatives, 7-aminoactinomycin D and these derivatives, Actinomycin D and these derivatives, ACMA(9-amino-6-chloro-2-methoxyacridine) and these derivatives, DAPI and these derivatives, Dihydroethidium and these derivatives, Ethidium bromide and these derivatives, EthD-1 and these derivatives, EthD-2 and these derivatives, Ethidium monoazide and these derivatives, Hexidium iodide and these derivatives, bisbenzimide(Hoechst 33258) and these derivatives, Hoechst 33342 and these derivatives, Hoechst 34580 and these derivatives, hydroxystilbamidine and these derivatives, LDS 751 and these derivatives, Propidium Iodide(PI) and these derivatives or Cy-dyes derivatives.

A fluorescent dye can be connected with at least one selected from the 5′ end region, the 3′ end region, and the middle region of oligonucleotide of probe and a fluorescent dye is labeled at said oligonucleotide by being bonded with or being replaced with a base of said oligonucleotide.

Another object of the present invention can be achieved by provide a real-time detection method of nucleic acid amplification, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said double-stranded nucleic acid into single strands by heating the aqueous buffer prepared in step i) up to 93° C. to 96° C; iii) annealing said pair of primers with said single strand by cooling the solution obtained in step ii) up to 50° C. to 57° C.; iv) replicating said single-stranded nucleic acid by heating the solution obtained from step iii) up to 70° C. to 74° C.; v) denaturing said replicated nucleic acid into single strands by heating the solution obtained in step iv) up to 93° C. to 96° C.; vi) annealing said fluorescent probe with said single-stranded nucleic acid by cooling the solution obtained in step v) up to 50° C. to 57° C.; vii) measuring an intensity of the fluorescence emitted from the solution obtained in step vi); vi) repeating more than one steps iv) through vii).

In ad method for a detection of an amplified nucleic acid, the step vii) can be simultaneously carried out with the step vi). Also said fluorescent probe can be hybridized with at least a portion of region which is inside the range of one (1) to fifteen (15) bases from the base on which 3′ end of said primer is combined.

Another object of the present invention can be achieved by providing a detection method of an initial amount of a nucleic acid in a sample, comprising the steps of i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase; ii) denaturing said double-stranded nucleic acid into single strands by heating the aqueous buffer prepared in step i) up to 93° C. to 96° C.; iii) annealing said pair of primers with said single strand by cooling the solution obtained in step ii) up to 50° C. to 57° C.; iv) replicating said single-stranded nucleic acid by heating the solution obtained from step iii) up to 70° C. to 74° C.; v) denaturing said replicated nucleic acid into single strands by heating the solution obtained in step iv) up to 93° C. to 96° C.; vi) annealing said fluorescent probe with said single-stranded nucleic acid by cooling the solution obtained in step v) up to 50° C. to 57° C.; vii) measuring an intensity of the fluorescence emitted from the solution obtained in step vi); viii) repeating more than one steps iv) through vii); ix) establishing a standard calibration curve which indicates the correlation between the log value of an initial amount of the nucleic acid and a threshold cycle shown by the performance of above steps i) through viii), by using a sample of which an initial amount of the nucleic acid is known; and x) detecting an initial amount of the nucleic acid based on the log value which corresponds to the threshold cycle obtained from the performance of said steps i) through viii), by referring to the standard calibration curve obtained in step ix).

A threshold cycle(C (T)) is an amplification frequency at the point of time when fluorescence intensity is detectable.

A proportional relationship is established between a log value of an initial amount of nucleic acids and the threshold cycle.

The linearity (R_{—}2) means a proportional relationship between the log value of an initial amount of a target nucleic acid and the threshold cycle obtained from real-time PCR.

As linearity (R_{—}2) of standard calibration curve closes to 1.0, an initial amount of a nucleic acid in an unknown sample can be correctly calculated. An initial amount of a nucleic acid in an unknown sample can be known by referring to a standard calibration curve.

The standard calibration curve can be obtained by measuring a log value of an initial amount of the nucleic acid in known sample and a threshold cycle shown by the performance of above steps i) through vi).

Another object of the present invention be achieved by providing a composition for the amplification of a nucleic acid which comprises i) a pair of primers for amplification of said nucleic acid; ii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iii) DNA polymerase; and iv) four (4) kinds of nucleotides.

The above composition for an amplification of a nucleic acid can be dried in vacuum.

Another object of the present invention can be achieved by providing a real-time detection method of the nucleic acid amplification, comprising the steps of i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase; ii) replicating a single-stranded cDNA by heating the aqueous buffer prepared in step i) up to 42° C. to 50° C.; iii) denaturing a primer for a reverse transcription and a reverse transcriptase from said single-stranded cDNA by heating the solution obtained from said step ii) up to 93° C. to 96° C.; iv) annealing the pair of primers with said single-stranded nucleic acid by cooling the solution obtained from said step iii) up to 50° C. to 57° C.; v) replicating said single-stranded nucleic acid by heating the solution obtained from step iv) up to 70° C. to 74° C.; vi) denaturing said replicated nucleic acid into single strands by heating the solution obtained from step v) up to 93° C. to 96° C.; vii) annealing said fluorescent probe with said single-strand nucleic acid by cooling the solution obtained from step vi) up to 50-57° C.; viii) measuring an intensity of the fluorescence emitted from the solution obtained in step vii); and ix) repeating more than one steps v) through viii).

Another object of the present invention can be achieved by providing a detection method of the initial amount of a nucleic acid in a sample, comprising the steps of i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase; ii) replicating a single-stranded cDNA by heating the aqueous buffer prepared in step i) up to 42° C. to 50° C.; iii) denaturing a primer for a reverse transcription and a reverse transcriptase from said single-stranded cDNA by heating the solution obtained from said step ii) up to 93° C. to 96° C.; iv) annealing the pair of primers with said single-stranded nucleic acid by cooling the solution obtained from said step iii) up to 50° C. to 57° C.; v) replicating said single-stranded nucleic acid by heating the solution obtained from step iv) up to 70° C. to 74° C.; vi) denaturing said replicated nucleic acid into single strands by heating the solution obtained from step v) up to 93° C. to 96° C.; vii) annealing said fluorescent probe with said single-strand nucleic acid by cooling the solution obtained from step vi) up to 50-57° C.; viii) measuring an intensity of the fluorescence emitted from the solution obtained in step vii); ix) repeating more than one steps v) through viii); x) establishing a standard calibration curve which indicates the correlation between the log value of an initial amount of the nucleic acid and a threshold cycle shown by the performance of above steps i) through ix), by using a sample of which an initial amount of the nucleic acid is known; and xi) detecting an initial amount of the nucleic acid based on the log value which corresponds to the threshold cycle obtained from the performance of said steps i) through ix), by referring to the standard calibration curve obtained in step ix).

Further object of the present invention can be achieved by providing a composition for the amplification of a nucleic acid which comprises i) a pair of primers for amplification of said nucleic acid; ii) a primer for reverse transcription iii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iv) DNA polymerase; v) a reverse transcriptase and iv) four (4) kinds of nucleotides.

A reverse transcriptase of the present invention means a RNA dependent DNA polymerase. The enzyme synthesizes cDNA (complementary DNA) from RNA template.

A reverse transcriptase is usually discovered in RNA viruses, which cause a tumor, or in the cell infected with RNA viruses. The enzyme is commonly used to the gene experiments for synthesizing DNA, which corresponds to mRNA.

Preferably a reverse enzyme in the present invention is selected from the group which consists of MMLV (Moloney Murine Leukemia Virus) reverse enzyme, AMV (Avian Myeloblastosis Virus) reverse enzyme, RAV-2 (Rous-Associated Virus Type 2) reverse enzyme or TTH (Thermus Thermophilus) reverse enzyme.

When a target nucleic acid of the present invention is RNA, cDNA should be first synthesized from the RNA. Primers can hybridize with said cDNA and replication can be started.

After a nucleic acid dissociates into a single-strand, an intensity of fluorescence can be detected by intercalation of a fluorescent dye.

Therefore, in the case that a target nucleic acid is RNA, an amplification of a nucleic acid can be detected quantitatively

The present invention is a novel method, which can detect and quantitatively analyze nucleic acids amplified by real-time PCR. When an intercalating dye of a probe of the present invention is intercalated into double-stranded nucleic acids, fluorescence of the dye can be detected. Intensity of fluorescence is proportional to an amount of amplified DNA and therefore quantitative analysis of amplified nucleic acids can be performed.

A method of the present invention can be used to monitor nucleic acids of various forms in vitro or in vivo. For example, the method can be used for PCR, hybridization, ligation, cleavage, recombination, synthesis, sequencing, mutation detection and biosensor for assessment of the concentration of lead, DNA, RNA and protein.

### [Advantageous Effects]

As mentioned above, by using the present invention, contrary to a method of real-time detection for the amplification of nucleic acids such as SYBR Green I, Foerst33228, Etidium Bromide (EtBr) etc., amplified nucleic acids of PCR can be detected more accurately, and time required for experiment is saved since it is not needed to measure the melting point of each products.

Hereinafter, the present invention will be described in detail with reference to the following examples. The examples are given only for illustration of the present invention and not to be limiting the present invention.

### DESCRIPTION OF DRAWINGS

The above objects and other advantages of the present invention will become more apparent by describing in detail a preferred embodiment thereof with reference to the attached drawings, in which:

FIGS. 1 to 2 is a schematic diagram of the process according to desired embodiment of the present invention.

FIG. 3 shows the molecular weight of an oligonucleotide labeled with DNA GREEN phosphoramidite at the 5′ end, which is measured by a mass spectrometer.

FIG. 4 shows the molecular weight of an oligonucleotide labeled with DNA GREEN phosphoramidite at the middle region, which is measured by a mass spectrometer.

FIG. 5 shows the intensity of fluorescence measured by a spectrophotofluorometer under light of 500 nm to 650 nm, after the hybridization of oligonucleotides labeled with DNA GREEN phosphoramidite at the 5′ end with complementary oligonucleotides.

FIG. 6 shows the fluorescence value of the meting curve measured by real-time PCR after the hybridization of oligonucleotides labeled with DNA GREEN phosphoramidite at the 5′ end with complementary oligonucleotides.

FIG. 7 shows the intensity variation of fluorescence of amplified products and a result of agarose gel electrophoresis after the performance of PCR of oligonucleotides labeled with DNA GREEN phosphoramidite at the 5′ end.

FIG. 8 shows the intensity variation of fluorescence of amplified products and a result of agarose gel electrophoresis after the performance of PCR of a positive control experiment group.

FIG. 9 shows the intensity variation of fluorescence of amplified products and a result of agarose gel electrophoresis after the performance of PCR of oligonucleotides labeled with DNA GREEN phosphoramidite at the middle region.

FIG. 10 shows the intensity variation of fluorescence of amplified products and a standard calibration curve after the performance of quantitative PCR of oligonucleotides labeled with DNA GREEN phosphoramidite at the 5′ end.

FIG. 11 shows the result of agarose gel electrophoresis of amplified products after the performance of quantitative PCR reaction of oligonucleotides labeled with DNA GREEN phosphoramidite at the 5′ end.

FIG. 12 shows the intensity variation of fluorescence of amplified products and a standard calibration curve after the performance of quantitative PCR of oligonucleotides labeled with DNA GREEN phosphoramidite at the middle region.

FIG. 13 shows the result of agarose gel electrophoresis of amplified products after the performance of quantitative PCR of oligonucleotides labeled with DNA GREEN phosphoramidite at the middle region.

FIG. 14 shows the intensity variation of fluorescence of amplified products and a result of agarose gel electrophoresis obtained after the performance of lambda DNA cross-contamination PCR of oligonucleotidea labeled with DNA GREEN phosphoramidite at the 5′ end.

FIG. 15 shows the intensity variation of fluorescence of amplified products and a result of agarose gel electrophoresis obtained after the performance of lambda DNA cross-contamination PCR of oligonucleotide labeled with DNA GREEN phosphoramidite at the middle region.

FIG. 16 shows the intensity variation of fluorescence of amplified products and result of agarose gel electrophoresis obtained after the performance of lambda DNA cross-contamination PCR of oligonucleotide labeled with DNA GREEN phosphoramidite at the 3′.

FIG. 17 shows the intensity variation of fluorescence of amplified products and a result of agarose gel electrophoresis obtained after thd perpormance of lambda DNA cross-contamination PCR of oligonucleotidea labeled with DNA GREEN phosphoramidite at the both ends (the 5′ end and 3′ end).

### BEST MODE

### EXAMPLE 1

### Extraction of Genomic DNA

AccuPrep Genomic DNA Extraction Kit was employed to extract genomic DNA of Mycobacterium tuberculosis, as follows.

A tubercle bacillus cultured in Ogawa medium (sodium glutamate 1 g, KH2PO4 3 g, distilled water 100 ml, chicken egg 200 ml, glycerin 6 ml, malachite green 2% solution 6 ml) and 5 ml of expectoration were added to mixture composed of 1 ml of TE(8.0) and 300 μl of Proteinase K(20 μg/μl).

then, 4 ml of lyses buffer (4M Urea) was added to the mixture. The mixture these obtained, was stirred for 20 minutes at 65° C. Binding buffer (7M GuanidineHCl) was added to the mixture. The mixture was stirred again for 20 minutes at 65° C. 2.75 ml of isopropanol was added to the solution and the mixture was centrifuged for 5 minutes in 2,500 rpm.

750 μl of the upper solution obrained in the above, was poured into each of binding column tube which contains glass filter and the solution contained in the column was centrifuged for 1 minutes in 12,000 rpm to remove effluent. Then 750 ul of washing buffer I (5M GuanidineHCl) was poured into the binding column and the mixture thus prepared, was centrifuged again for 1 minute in 13000 rpm to remove effluent.

750 ul of washing buffer II (20 mM NaCl) was added to the mixture obtained in the above contained in the binding column and then centrifuged for 1 minute in 12,000 rpm to eliminate effluent. And said column was centrifuged for 2 minutes in 12,000 rpm again to remove the washing buffer left in the binding column tube.

The binding column which contains the glass filter, was put into 1.5 ml tube. 100 μl of an elution buffer(10 mM TrisHCl) was poured into said 1.5 ml tube which contains the binding column and the glass filter. The tube was left for 5 minutes in room temperature. Then, the mixture thus prepared was centrifuged for 2 minutes in 12,000 rpm again to remove effluent. Collected DNA in Example 1 was used in Examples 4 through 9.

### EXAMPLE 2

### Synthesis of DNA Green Phosphoramidite

A phosphoramidite containing fluorescent material of chemical formula 1, was prepared according to the process discribed in U.S. Pat. No. 6,348,596 and U.S. Pat. No. 6,080,868. In this specification, the phosphoramidite containing fluorescent material employed in this invention, is represented as DNA GREEN phosphoramidite. A probe used in the present invention was prepared by labeling with the DNA GREEN phodphoramidite at a desired location.

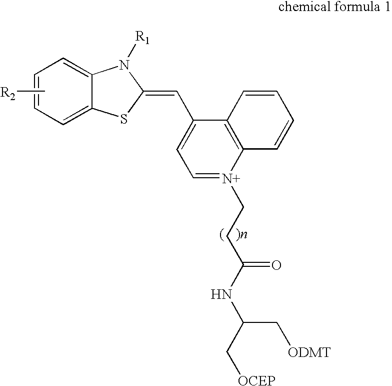

Wherein, n is 2, 3, 4 or 5. R₁ is one of —CH₃, —CHCH₂CH₂OH, —CH₂CH₂CH₂CH₂CH₂CH₂OH, —CH₂CH₂CO₂H and —CH₂CH₂Br. R₂ is H, OH, or NO₂. CEP represents cyano ehtoxy phosphoramidite and DMT represents dimethoxytrityl.

### EXAMPLE 3

### Design of a Primer and a Probe

RpoB gene of mycobacterium tuberculosis was employed in this Example to produce a primer and a probe of the present invention.

The oligonucleotides, which can be hybridized with the above RpoB gene, were designed by using Beacon Designer 2.1 (PREMIER Biosoft International co.), a program for designing primer and probe (Seq. ID Nos. 1 to 5, Seq. ID Nos. 8 to 17, Seq. ID Nos. 19 to 22). The oligonucleotides prepared, are represented in Table 1 and Table 2.

Seq. ID Nos. 8 through 12 of Table 1 and Table 2, are fluorescent probes labeled with DNA GREEN phosphoramidite at the 5′ end and the numbers in Contents column, indicate the number of bases. For example, 29 represents that the oligonucleotide consists of total 29 bases and is labeled with DNA GREEN phosphoramidite at the 5′ end of 28th base from the 3′ end through the chemical substitution or addition at site of 29th base.

Seq. ID Nos. 13 to 17 inclusive, are oligoncleotides labeled with DNA GREEN phosphoramidite at the middle region of a base sequence and the numbers in Contents column in Table 1, indicate the number of bases. For example, 28 means that the oligonulceotide consists of total 28 bases and is labeled with DNA GREEN phosphoramidite at the 10th base apart from the 3′ end of the probe through the chemical substitution or addition.

Seq. ID No. 21 is an oligonucleotide labeled with DNA GREEN phosphoramidite at the 3′ end and the number in Contents column in Table 2, indicates the number of base. For example, 23 means that an oligonucleotide consists of total 23 bases and is labeled with DNA GREEN phosphoramidite at the 3′ end of 22nd base from the 5′ end through the chemical substitution or addition.

Seq. ID No. 22 is an oligoncleotide labeled with DNA GREEN phosphoramidite at the 5′ end and the 3′ end and the number in contents indicates the size of base. For example, 24 means that an oligonucleotide consists of total 24 bases wherein the oligonucleotide is labeled with DNA GREEN phosphoramidite at the 5′ end and 3′ end of 22 bases by being bonded with or replaced with 1st and 24th bases.

Meanwhile, two oligonucleotides are needed for nucleic acid amplification as primers. The oligonucleotides are called a forward primer and a reverse primer. The F means forward primer, the R means reverse primer in Table 1 and Table 2. In Table 1 and Table 2, Seq. ID Nos. 1 to 2, Seq. ID Nos. 3 to 4 and Seq. ID Nos. 6 to 7 and Seq. ID Nos. 19 to 20 were used as primers.

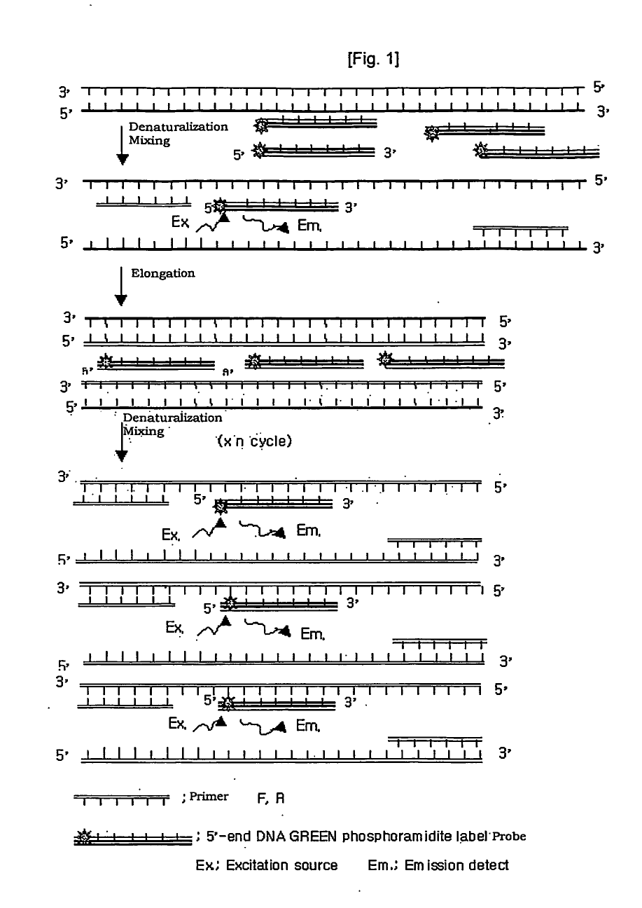

At least one of Seq. ID Nos. 8 to 17, Seq. ID No. 20, Seq. ID No. 21 and Seq. ID No. 22 was used as a fluorescent probe. The probe comprises an oligonucleotide of which base sequence is complementary with at least a part of said nucleic acid. The probe can be hybridized with at least a portion of region, which is inside the range of one (1) to fifteen (15) bases from the base of target nucleic acid on which 3′ end of said primer is combined. Its schematic diagram is represented in FIG. 1.

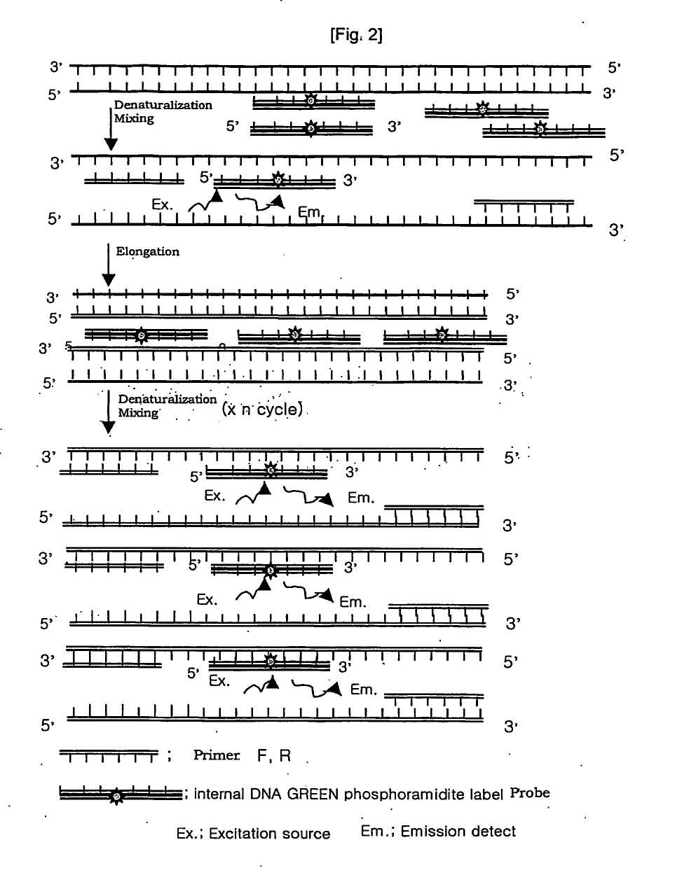

In Seq. ID Nos. 13 to 17, the 10th base from 3′ end of an oligonucleotide is replaced with DNA GREEN phosphoramidite and the oligonucleotide can hybridize with complementary base sequence in amplified DNA. Its schematic diagram is represented in FIG. 2.

In Seq. ID No. 21 and Seq. ID No. 22, the 3′ end of Seq. No. 21 and the 3′ end and 5′ end of Seq. No. 22 were label with DNA GREEN phosphoramidite and said base sequences were designed to hybridize with amplified DNA.

Meanwhile, conventional methods for detecting a exact amount of nucleic acid in a sample by analyzing samples obtained from each cycles of real time PCR are such as Taqman (Holland et al., 1991, Proc. Natl. Acad. Sci. USA 88:7276-7280; Lee et al., 1993, Nucleic Acids Res. 21:3761-3776), Molecular Beacon (Tyagi & Kramer 1996, Nature Biotech. 14:303-309, U.S. Pat. No. 5,119,801, U.S. Pat. No. 5,312,728) etc. Seq. ID No. 5 in Table 1 and Table 2 was used in a Molecular Beacon method.

Table 3 is a list of fluorescent dyes classified according to a wavelength. Each fluorescent dye has particular excitation wavelength and emission wavelength. The values in Table 3 mean an optimal excitation wavelength.

### Oligontide Sequences

TABLE 1

| | | | |
|---|---|---|---|
| Seq. | | | |
| ID | Con- | | |
| Nos. | tent | Sequence | remark |

| | | | |
|---|---|---|---|
| 1 | Primer | 5′ agt gca aag aca agg aca | Oligonucleo- |
| | F1 | tga-3′ | tide for nu- |
| | | | cleic acid |
| | | | amplification |
| 2 | Primer | 5′ ttc tcg gtc atc atc ggg | Oligonucleo- |
| | R1 | aa-3′ | tide for nu- |
| | | | cleic acid |
| | | | amplification |
| 3 | Primer | 5′ gat gtc gtt gtc gtt | Oligonucleo- |
| | F2 | ctc-3′ | tide for de- |
| | | | tection of |
| | | | probe of con- |
| | | | trolled group |
| 4 | Primer | 5′ acc gtc tga ctc ttg | Oligonucleo- |
| | R2 | atc-3′ | tide for de- |
| | | | tection of |
| | | | probe of con- |
| | | | trolled group |
| 5 | Probe | 5′ cgc gat gtc acc gcc gag | Probe of the |
| | 1 | ttc atc aac aaa tcg cg-3′ | controlled |
| | | | group, la- |
| | | | beled with |
| | | | Fluoresein at |
| | | | the 5′ end |
| | | | and Dabcyl at |
| | | | the 3′ end |
| 6 | Primer | 5′ acc tca ttt tca tgt ccg | Oligonucleo- |
| | F3 | gtc agc-3′ | tide for am- |
| | | | plification |
| | | | of Lambda |
| | | | 100 bp |
| 7 | Primer | 5′ ggc aga gct gaa aga gga | Oligonucleo- |
| | R3 | gct tga-3′ | tide for am- |
| | | | plification |
| | | | of Lambda |
| | | | 100 bp |
| 8 | 29 | 5′ *cc atg aac acc gtc tga | labeled with |
| | | ctc ttg atc tc-3′ | DNA GREEN |
| | | | phosphorami- |
| | | | dite at the |
| | | | 5′ end |
| 9 | 27 | 5′ *cc atg aac acc gtc tga | labeled with |
| | | ctc ttg atc-3′ | DNA GREEN |
| | | | phosphorami- |
| | | | dite at the |
| | | | 5′ end |
| 10 | 25 | 5′ *cc atg aac acc gtc tga | labeled with |
| | | ctc ttg a-3′ | DNA GREEN |
| | | | phosphorami- |
| | | | dite at the |
| | | | 5′ end |
| 11 | 23 | 5′ *cc atg aac acc gtc tga | labeled with |
| | | ctc tt-3′ | DNA GREEN |
| | | | phosphorami- |
| | | | dite at the |
| | | | 5′ end |
| 12 | 21 | 5′ *cc atg aac acc gtc tga | labeled with |
| | | ctc-3′ | DNA GREEN |
| | | | phosphorami- |
| | | | dite at the |
| | | | 5′ end |
| 13 | 28 | 5′ cca tga aca ccg tct gac | labeled with |
| | | *ct tga tct c-3′ | DNA GREEN |
| | | | phosphorami- |
| | | | dite at the |
| | | | middle region |
| 14 | 26 | 5′ cca tga aca ccg tct g*c | labeled with |
| | | tct tga tc-3′ | DNA GREEN |
| | | | phosphorami- |
| | | | dite at the |
| | | | middle region |
| 15 | 24 | 5′ cca tga aca ccg tc* gac | labeled with |
| | | tct tga-3′ | DNA GREEN |
| | | | phosphorami- |
| | | | dite at the |
| | | | middle region |
| 16 | 22 | 5′ cca tga aca ccg *ct gac | labeled with |
| | | tct t-3′ | DNA GREEN |
| | | | phosphorami- |
| | | | dite at the |
| | | | middle region |
| 17 | 20 | 5′ cca tga aca c*g tct gac | labeled with |
| | | tc-3′ | DNA GREEN |
| | | | phosphorami- |
| | | | dite at the |
| | | | middle region |

### Oligonucleotide Sequences

TABLE 2

| | | | |
|---|---|---|---|
| 18 | D.G- | 5′ ccc ttc agt ggg tac ttg tgg | Base se- |
| | ph- | cag act gag aac tag agt ggc | quence com- |
| | com | c-3′ | plementary |
| | | | with Seq. |
| | | | ID Nos. 8 |
| | | | to 17 |
| 19 | 21 | 5′ caa gag tca gac ggt gtt | Oligonucle- |
| | | ca-3′ | otide for |
| | | | nucleic |
| | | | acid ampli- |
| | | | fication |
| 20 | 22 | 5′ ttg tcg gtg gac ttg tca | Oligonucle- |
| | | at-3′ | otide for |
| | | | nucleic |
| | | | acid ampli- |
| | | | fication |
| 21 | 23 | 5′ tga ctt ccc gat gat gac cga | labeled |
| | | g*-3′ | with DNA |
| | | | GREEN phos- |
| | | | phoramidite |
| | | | at the 3′ |
| | | | end |
| 22 | 24 | 5′ *tg act tcc cga tga tga ccg | labeled |
| | | ag*-3′ | with DNA |
| | | | GREEN phos- |
| | | | phoramidite |
| | | | at the 3′ |
| | | | end and 5′ |
| | | | end |

### List of Fluorescent Dye Classified According to Wavelength

TABLE 3

| Excitation (nm) | Emission (nm) | Recommended Fluorophores |
|---|---|---|
| 490 ± 10 | 520 ± 10 | FAM, SYBR Green I, Fluorescein |
| 510 ± 5 | 530 ± 5 | DNA GREEN phosphoramidite |
| 525 ± 10 | 550 ± 10 | HEX, TET, VIC, JOE |
| 530 | 620 | EtBr |
| 560 ± 10 | 570 ± 10 | TAMRA, Cy3, Rhodamine red |
| 585 ± 10 | 610 ± 10 | Texas Red, ROX |
| 625 | 640 | LC640 |
| 640 ± 10 | 660 ± 10 | Cy5 |
| 675 ± 10 | 700 ± 10 | Cy5,5, LC705 |

### EXAMPLE 4

### Investigation of Oligonucleotide Labeled with DNA GREEN Phosphoramidite

Oligonucleotide sequences of Table 1. and Table 2. were prepared by Nucleic Acid Synthesis System EXPEDITE (Perseptive Biosystems Co.). The Molecular weights of oligonucleotides prepared by the system, were measured by using Axima-LNR (Maldi-Tof, SHIMADZU Co.) which is a polymer mass spectrometer.

Maldi-Tof (Matrix-Assorsted Laser Desruption/Ionozation Time of Flight) is an apparatus for measuring a modification rate of a depurination oligonucleotide, an N−1 failed oligonucleotide and various modificated oligonucleotides by analyzing the molecular weight measured actually and the molecular weight expected from the weight of an oligonucleotide.

At least, one of oligonucleotides of Seq. ID Nos. 8 through 12 in Table 1 and Table 2, was used in the present Example.

As indicated in FIG. 3, the molecular weight of Seq. ID No. 8 measured by a polymer mass spectrometer, shows the analogousness between the expected molecular mass (8941.2 g/mole) and the measured molecular mass (8914.6 g/mole). The X-axis represents a measured molecular weight divided by a charge of oligonucleotide. The Y-axis represents oligonucleotide intensity measured by a unit of percentages.

Peak 1 indicates the molecular weight of target oligonucleotide. Pick 2 indicates the measured molecular weight divided by charge value 2.

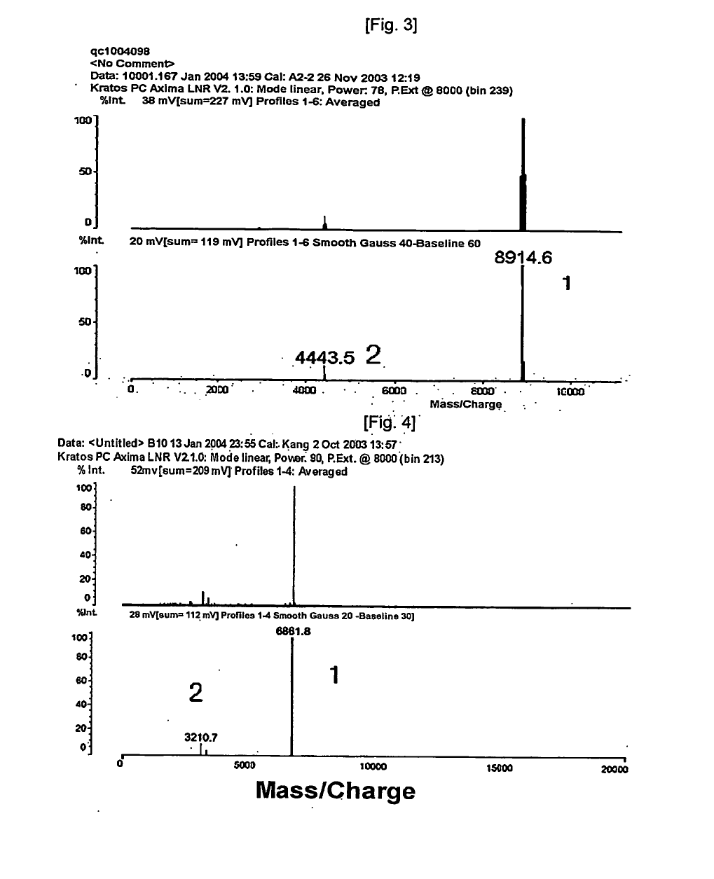

At least, one of oligonucleotides of Seq. ID Nos. 13 through 17 in Table 1 and Table 2, was used in the present Example. As indicated in FIG. 4, the molecular weight of Seq. ID NO. 16 measured by a polymer mass spectrometer, shows the analogousness between the expected molecular mass (6868.0 g/mole) and the measured molecular mass (6861.8 g/mole). The X-axis represents a measured molecular weight divided by a charge of oligonucleotide. The Y-axis represents oligonucleotide intensity measured by a unit of percentages.

Peak 1 indicates the molecular weight of target oligonucleotide. Pick 2 indicates the measured molecular weight divided by charge value 2.

An excitation wavelength and an emission wavelength of the oligonucleotide labeled with DNA GREEN phosphoramidite, of which the mass and the purity was measured by a polymer mass spectrometer, were measured again by a spectrofluorophotometer (RF-5301PC, SHIMADZU Co.).

Also, the variation of a fluorescence intensity of oligonucleotide when DNA GREEN phosphoramidite intercalates into a double-strand nucleic acid, was measured by using an Opticon™ real time PCR (MJ Reasearch). The mass and purity of said oligonucleotide were measured by a polymer mass spectrometer.

At least, one of oligonucleotides of Seq. ID Nos. 8 through 17 in Table 1 and Table 2, was used in the present Example. Seq. ID No. 18 which is complementary with the target nucleic acid, was used to measure the variation of the fluorescence intensity when DNA GREEN phosphoramidite intercalates into a double-strand DNA.

Two 15 ml tubes were prepared to measure a excitation wavelength and an emission wavelength of the oligonucleotide labeled with DNA GREEN phosphoramidite by using a Spectrofluorophotometer.

2.9 ml of TEM buffer (10 mM TrisHCl, 1M EDTA, pH 8.0, final 3.5 mM MgCl2) and 100 μl of Seq. ID No. 10 (10 pmole/μl) were mixed in tube 1. 2.8 ml of TEM buffer (10 mM TrisHCl, 1 mM EDTA, pH 8.0, final 3.5 mM MgCl2), 100 μl of Seq. ID No. 10 (10 pmole/μl) and 100 μl and Seq. ID No. 18 (10 pmole/μl), were mixed in tube 2. The final volume of the solution in said tubes should become 3 ml and the nucleic acids in the tubes were denatured for 5 minutes at 94° C. respectively. After the procedure, the tube 1 was left on ice.

Nucleic acids of tube 2 were hybridized by slowly cooling the tube up to the room temperature. The excitation wavelength and emission wavelength of the oligonucleotide labeled with DNA GREEN phosphoramidite, were scanned by a spectrofluorophotometer in condition of 475 nm/450 nm-800 nm to 475 nm/500 nm-600 nm, 480 nm/450 nm-800 nm to 480 nm/500 nm-600 nm, 485 nm/450nm-800 nm to 485 nm/500 nm-600 nm, 490 nm/450 nm-800 nm to 490 nm/500 nm-600 nm, 495 nm/450 nm-800 nm to 495 nm/500 nm-600 nm, 500 nm/450 nm-800 nm to 500 nm/500 nm-600 nm, 505 nm/450 nm-800 nm to 505 nm/500 nm-600 nm, 510 nm/450 nm-800 nm to 510nm/500 nm-600 nm, 515 nm/450 nm-800 nm to 515 nm/500 nm-600 nm, 520 nm/450 nm-800 nm to 520 nm/500 nm-600 nm, and 525 nm/450 nm-800 nm to 525nm/500 nm-600 nm.

In these measurement, the excited fluorescence intensity was increased as the emission wavelength was increased regradless the addition of the complementary. The excited fluorescence intensity when a complementary oligonucleotide was added, was larger than the intensity when a complementary oligonucleotide was not added.

A change of the fluorescent intensity when DNA GREEN phosphoramidite was intercalated into double strand nucleic acid, and the shifting effect of the excited fluorescence wavelength, were detected. A preferable excited fluorescent intensity was also detected in the range of emission wavelength of 505 nm to 515 nm. The preferable excited wavelength, was 530 nm to 540 nm.

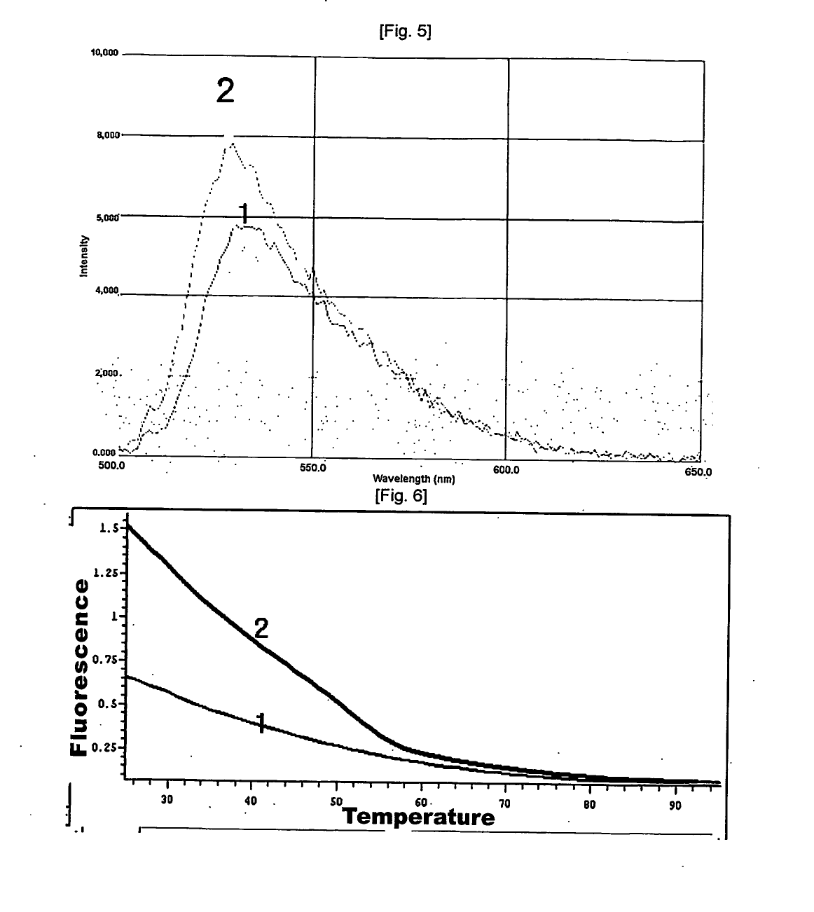

FIG. 5 shows a fluorescent intensity of Seq. ID No. 10 or a hybrid of Seq. ID No. 10 and 18 when the emission wavelength is 505 nm and an excitation wavelength is in the range of 500 nm to 650 nm. The excited fluorescence intensity, was increased from 530 nm through 500 nm and was decreased until 650 nm.

The X-axis of FIG. 5 represents a scanning wavelength for measuring excited fluorescence intensity and the Y-axis represents the fluorescence intensity measured in each scanning wavelength. Peak 1 indicates the excited fluorescence intensity when emission wavelength is 505 nm for Seq. ID No.10. Peak 2 is a graph of excited fluorescence intensity when emission wavelength is 505 nm for the hybrid of Seq. ID No.10 and 18.

Two tubes were prepared for real time DNA amplification. The variation of a fluorescence intensity of probe when DNA GREEN phosphoramidite intercalates into a double-strand nucleic acid, was measured.

49 μl of TEM buffer (10 mM TrisHCl, 1 mM EDTA, pH 8.0, final 3.5 mM MgCl2) and 1 μl of Seq. ID No. 10 (10 pmole/μl), were mixed in tube 1. 48 μl of TEM buffer (10 mM TrisHCl, 1 mM EDTA, pH8.0, final 3.5 mM MgCl2) and 1 μl of Seq. ID No. 10 (10 pmole/μl) and Seq. ID No. 18 (10 pmole/μl), were also mixed in tube 2. The final volume of the solution in these tubes should become 50 μl, respectively, and real time DNA amplification apparatus was operated.

The condition of real time PCR, was that pre-denaturalization for 5 minutes at 94° C., pre-cooling for 5 minutes at 25° C., and scanning the fluorescence intensity whenever 1° C. was increased during PCR from 25° C. to 95° C.

In the measurement, the fluorescence intensity was decreased as the reaction temperature was increased in both conditions that a complementary oligonucleotide was added or not.

Also, the measured fluorescence intensity when a complementary oligonucleotide was added, was larger than the intensity measured under the condition that complementary oligonucleotide was not added. The difference between them represents the changing effect of fluorescence intensity by intercalation.

As indicated in FIG. 6, the melting curve of the Seq. ID No. 10 or the hybrid of Seq. ID Nos.10 and 18, are illustrated from 25° C. to 94° C. by real time PCR. The X-axis represents a reaction temperature and the Y-axis represents a fluorescence intensity measured in each temperature. Peak 1 indicates the measured fluorescence intensity of the Seq. ID No. 10 at the reaction temperature. Peak 2 indicates the intensity of the hybrid of Seq. ID Nos. 10 and 18 at the reaction temperature.

### EXAMPLE 5

### The PCR Reaction for the Probe Labeled DNA GREEN Phosphoramidite at the 5′ End

The primer is a specific base sequence for an amplification of target nucleic acid in real-time PCR. The probe is composed of bases which is complementary to a target nucleic acid, and hybridized with target nucleic acid adjacent to a primer. The probe is labeled with a fluorescent dye at least one of regions in the 5′ end, the 3′ end or the middle of base sequences (Table 1 and Table 2).

In a positive control group, the probe of the Molecular Beacon method, which is a conventional method for calculating correct concentration of nucleic acids in a sample by analyzing samples obtained from each cycles of real-time PCR, was used in real-time PCR. Therefore, the data of the positive control group, was used for a criterion for establishing an availability of using the probe labeled with DNA GREEN phosphoramidite.

Clen Taq polymerase is a polymerase of which activity and heat stability is increased remarkably by eliminating the 5′ exonuclease activity through the 5′ deletion of the gene which encodes Taq polymerase. This Clen Taq polymerase can remove the non-specificity which is occured due to the 5′ exonuclease activity of Taq polymerase to the probe labeled with fluorescent dye at the 5′ end in the real-time PCR.

In the present Example, Seq. ID No. 1 and 2 of Table 1 and Table 2 were used as primers and at least one of Seq. ID No. 8 through 12 were used as probes. Seq. ID No. 3 and 4 were used as a primer and Seq. ID No.5 was used as a probe for a positive control group.

20 μl of reaction buffer was prepared in each tubes to perform real-time PCR by using said primer and primer.

The buffer was prepared by following procedures. 10 mM dNTPs (2.5 mM dATP, 2.5 mM dGTP, 2.5 mM dCTP, 2.5 mM dTTP, respectively) and 20 mM of MgCl₂ were added into the tubes. The final concentration of the 10× reaction buffer, dNTPs and MgCl2 was 2 mM, 2.5 mM and 3 mM respectively. And Seq. ID Nos. 1 and 2, primers for an amplification of target nucleic acid, were added into the tubes. The final concentration of the primers was 0.5 uM.

Then, Seq. ID Nos. 8 through 12, the probes labeled with DNA GREEN phosphoramidite at the middle region, were added to the tubes. The final concentration of the probes was 0.5 uM, 1.0 uM, 2.5 uM and 5.0 uM respectively. Clen Taq polymerase (BIONEER Co.) was added into each tubes to get the concentration to be 0.2 U (unit). 2 μl of refined tubercle DNA of Example 1 was added to each tubes and theses tubes were filled with distilled water to get final volume to be 20 μl. After stirring the solution of the tubes, they were spun down by microcentrifuge.

For a positive control group, 2 μl of 10× reaction buffer (20 mM Tris-HCl, 100 mM KCl, pH 9.0), 2 μl of 10 mM dNTPs (2.5 mM dATP, 2.5 mM dGTP, 2.5 mM dCTP, 2.5 mM dTTP, respectively) and 20 mM of MgCl₂ were added to the tubes. The final concentration of the 10× reaction buffer, dNTPs and MgCl₂ was 2.5 mM respectively. Then, Seq. ID Nos. 3 and 4, primers for an amplification of target nucleic acid, were added to get the concentration to be 0.5 uM. Seq. ID No. 5, a probe labeled with fluorescence dye, was added to get the final concentration to be 0.25 uM.

Clen Taq polymerase (BIONEER Co.) was added to each tubes to get the concentration to be 0.2 U (unit). 2 μl of refined tubercle DNA in Example. 1 was added to each tubes. Then, the tubes were filled with distilled water to get the final volume to be 20 μl and were spun down by microcentrifuge.

Three steps of real-time PCR was carried out by using PCR machine (Opticon™, MJ Co.). In above PCR, pre-denaturing for 5 min. at 95° C., denaturing for 30 sec at 95° C., annealing for 60 sec at 50° C. and elongation for 40 sec at 72° C., were carried out for 45 times.

Fluorescence intensities of every annealing step were measured and an amplification curve of products of real-time PCR was established by using the measured data.

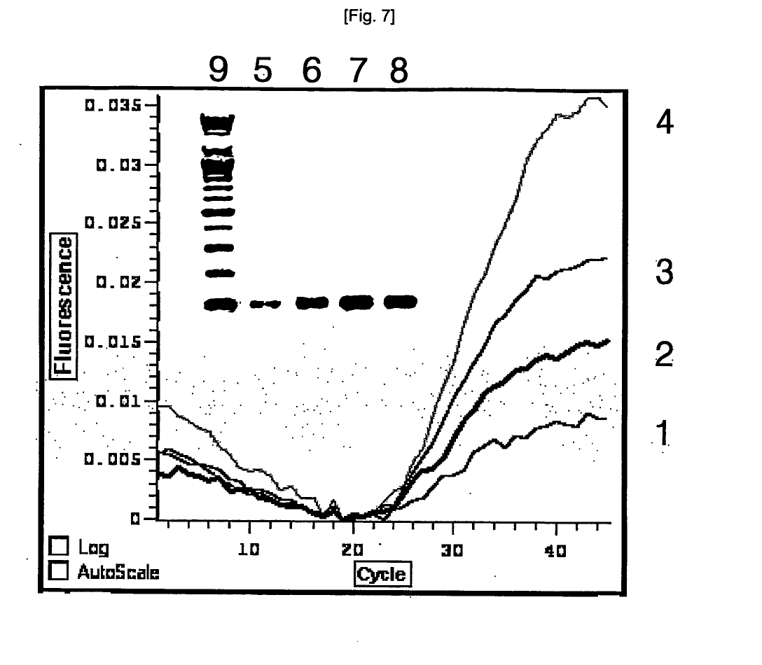

FIG. 7 indicates the result of real time PCR for Seq. ID No. 8 when the concentrations of nucleotides were 0.5 uM, 1.0 uM, 2.5 uM and 5.0 uM. The reaction result showed an increase of fluorescence intensity as reaction cycles was repeated.

The X-axis of FIG. 7 represents a PCR reaction cycle and the Y-axis is measured fluorescence intensity. Peak 1 to Peak 4 are graphs for the fluorescence intensity of each reaction cycles when concentration of oligonucleotide labeled with DNA GREEN phosphoramidite at the 5′ end were 0.5 uM, 1.0 uM, 2.5 uM and 5.0 uM.

As indicated in FIG. 8, fluorescence intensity of positive control was increased as reaction cycles were repeated. The X-axis is a PCR reaction cycle and the Y-axis is measured fluorescence intensity. Peaks 1 to 2 are graphs for the fluorescence intensity of each reaction cycles when the concentration of the probe was 0.25 uM.

PCR product obtained by above reaction was certified by gel electrophoresis. 2 g of agarose (BIONEER Co.) was put into a beaker and 0.5× TBE was added. Final volume of the mixture was 100 ml. The mixture was heated and stirred until correctly melted.

4 μl of EtBr(10 mg/ml) was added when the mixture was cooled until 60° C. The mixture was put into a casting tray with comb and left for 30 minutes in room temperature to make the mixture hard. The hardened gel was put into an AgaroPower™ (BIONEER Co.) which is an electrophoresis chamber. The chamber was filled with 0.5× TBE buffer for electrophoresis until the gel was sunk.

5 μl of mixture of PCR product and 1 μl of loading buffer were mixed and loaded to a well in agarose gel by using pipette. After electrophoresis, the gel was put on a UV transilluminator and taken a picture by Imager III™ which is a digital camera. In the present invention, size of amplification product was 127 base pairs (bp) and the size of amplification product of positive control was 150 base pairs (bp).

The result of gel electrophoresis is indicated in FIG. 7 and FIG. 8. In FIG. 7, lane 5 is a result of a gel electrophoresis of 0.5 uM of oligonucleotide labeled with DNA GREEN phosphoramidite at the 5′ end, lane 6 is the result of a gel electrophoresis of 1.0 uM of oligonucleotide labeled with DNA GREEN phosphoramidite at the 5′ end, lane 7 is a result of a gel electrophoresis of 2.5 uM of oligonucleotide labeled with DNA GREEN phosphoramidite at the 5′ end and lane 8 is a result of a gel electrophoresis of 5.0 uM oligonucleotide labeled with DNA GREEN phosphoramidite at the 5′ end.

Lane 9 represents 100 bp size marker. In FIG. 8, lane 3 to 4 is the result of gel electrophoresis of positive control in which the concentration of the probe was 0.25 uM. Lane 5 represents 100 bp size marker.

### EXAMPLE 6

### PCR Using a Probe Labeled DNA GREEN Phosphoramidite at the Middle Region

In the present Example, Seq. ID No.1 and 2 in Table 1 and Table 2 was used as a primer, at least one of oligonucleotides of Seq. ID No.13 to 17 was used as a probe.

20 μl of reaction buffer was prepared in each tubes to perform PCR by using said each primer and primer.

The buffer was prepared by following procedures.

2 μl of 10× reaction buffer (200 mM Tris-HCl, 100 mM KCl, pH 9.0), 2 μl of 10 mM dNTPs (2.5 mM dATP, 2.5 mM dGTP, 2.5 mM dCTP, 2.5 mM dTTP, respectively) and 20 mM of MgCl2 were added to the tubes to get the final concentration of the mixture to be 1.5 mM, 2 mM and 2.5 mM respectively. And Seq. ID Nos. 1 and 2, primers for an amplification of target nucleic acid, were added to get the concentration to be 0.5 uM.

Afterward, Seq. ID Nos. 13 to 17, a probe labeled with DNA GREEN phosphoramidite at the middle region, was added to the tubes to get the concentration to be 0.5 um, 1.0 uM, 2.5 uM and 5.0 uM. And Clen Taq polymerase (BIONEER Co.) was added to each reaction tubes to become 0.17 U (unit). And 1.5 μl of refined tubercle DNA in Example 1 was added to each tube. The tubes were filled with distilled water to get final volume to be 20 μl and were spun down by micro centrifuge.

Afterward, real-time PCR was carried out by using PCR machine (Opticon™, MJ Co.). In above PCR, pre-denaturing for 5 min. at 94° C., denaturing for 30 sec at 95° C., annealing for 50 sec at 56° C. and elongation for 40 sec at 72° C., were carried out for 46 times.

Fluorescence intensities of every annealing step were measured and an amplification curve of products of real-time PCR was established by using the measured data.

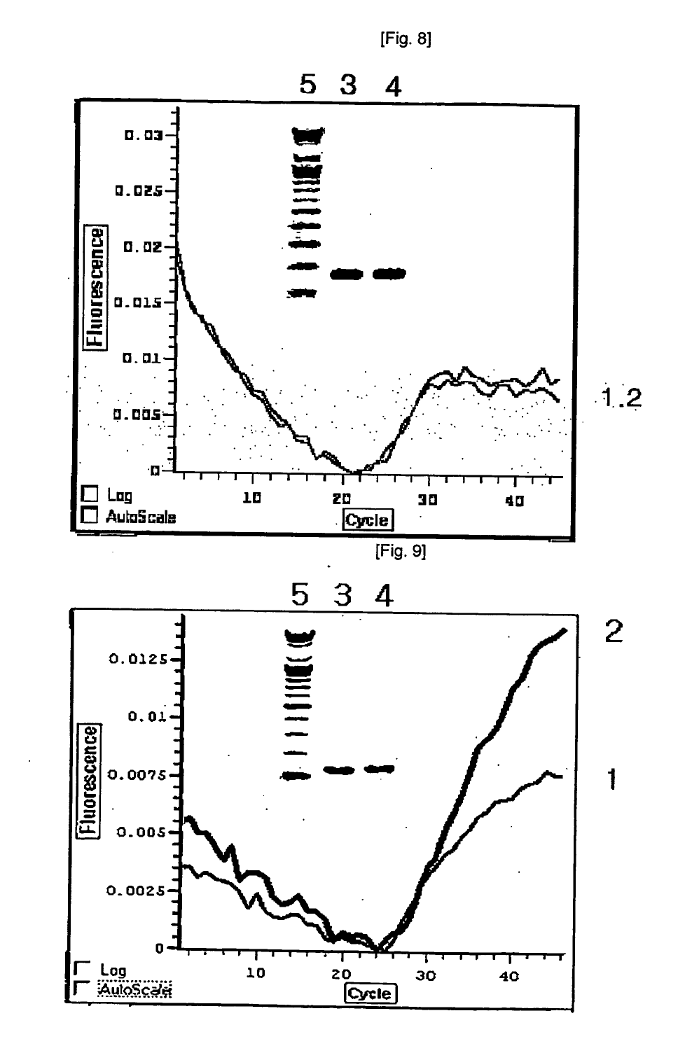

FIG. 9 shows the result of real time PCR of Seq. ID No. 16 when the concentrations of oligonucleotides were 0.5 uM and 1.0 uM and the concentration of MgCl2 was 1.5 mM. The fluorescence intensity was increased as reaction cycles were repeated.

In FIG. 9, the X-axis represents PCR reaction cycles and the Y-axis represents measured fluorescence intensity. Peak 1 is a graph of the fluorescence intensity of each reaction cycles when the concentrations of oligonucleotides labeled with DNA GREEN phosphoramidite at the middle region were 0.5 uM. Peak 2 is a graph of the fluorescence intensity of each reaction cycles when the concentrations of oligonucleotides labeled with DNA GREEN phosphoramidite at the middle region were 1.0 uM.

PCR product was confirmed by performing a gel electrophoresis, which is prepared by a method of Example 4. Size of the amplified products obtained by using a primer labeled with DNA phosphoramidite at the middle region, was 127 bp.

In FIG. 8, Lane 3 is a result of a gel electrophoresis of real time PCR products of 0.5 uM of oligonucleotide labeled with DNA phosphoramidite at the middle region and Lane 4 is a result of a gel electrophoresis of real time PCR products of 1.0 uM of oligonucleotide labeled with DNA phosphoramidite at the middle region. Lane 5 represents 100 bp size marker.

### EXAMPLE 7

### Fixed Quantity PCR Using a Probe Labeled with DNA GREEN Phosphoramidite at the 5′ End of Base Sequence

In a negative control, distilled water was used for PCR instead of target nucleic acids. A negative control shows a level of contamination, which is created by external causes during PCR indirectly.

Fixed quantity real time PCR was carried out for tubercle DNA in order of concentration according to the reaction condition in Example 4. Seq. ID No.1 and 2 produced in Example 2 were used as primers and at least one of oligonucleotides of Seq. ID No.8 to 12 was used as probes.

20 μl of reaction buffer was prepared in each tubes to perform PCR by using said each primer and primer.

The buffer was prepared by following procedures.

2 μl of 10× reaction buffer (200 mM Tris-HCl, 100 mM KCl, pH 9.0), 2 μl of 10 mM of dNTPs (2.5 mM dATP, 2.5 mM dGTP, 2.5 mM dCTP, 2.5 mM dTTP, respectively) and 20 mM of MgCl2 were added to the tubes to get the concentration of the mixture to be finally 2 mM. And Seq. ID Nos. 1 and 2, a primer for amplification of target nucleic acid, were added to get the final concentration to be 0.5 uM.

Seq. ID No. 10, a probe labeled with DNA GREEN phosphoramidite at the 5′ end, was added to the tubes to get the concentration to be 5.0 uM. And Clen Taq polymerase (BIONEER Co.) was added to each reaction tubes to become 0.2 U (unit). The tubes were filled with distilled water to get final volume to be 20 μl and spun down by micro centrifuge. Distilled water was used as a negative control.

Afterward, real-time PCR was carried out by using PCR machine (Opticon™, MJ Co.). In above PCR, pre-denaturing for 5 min. at 94° C., denaturing for 30 sec at 95° C., annealing for 60 sec at 55° C. and elongation for 40 sec at 72° C., were carried out for 45 times.

Fluorescence intensities of every annealing step were measured and an amplification curve of products of real-time PCR was established by using the measured data.

Afterward, the amplification curve was changed logarithmically and the threshold cycle[C (T)] was decided. Standard calibration curve was made and the linearity of fixed quantity PCR was decided.

PCR products were confirmed by performing a gel electrophoresis, which is prepared by a method of Example 4. Size of the amplified products obtained by using a primer labeled with DNA GREEN phosphoramidite at the 5′ end, was 127 bp.

As indicated in FIG. 10, left graph is the said log of amplification curve deciding the threshold cycle(C (T)). Right graph is the standard calibration curve of serially diluted tubercle DNA according to the threshold cycle[C (T)] curve.

Compared with the negative control, left graph shows an increase of fluorescence intensity as PCR reaction repeated. Right graph shows that the linearity (R_{—}2) is 0.997.

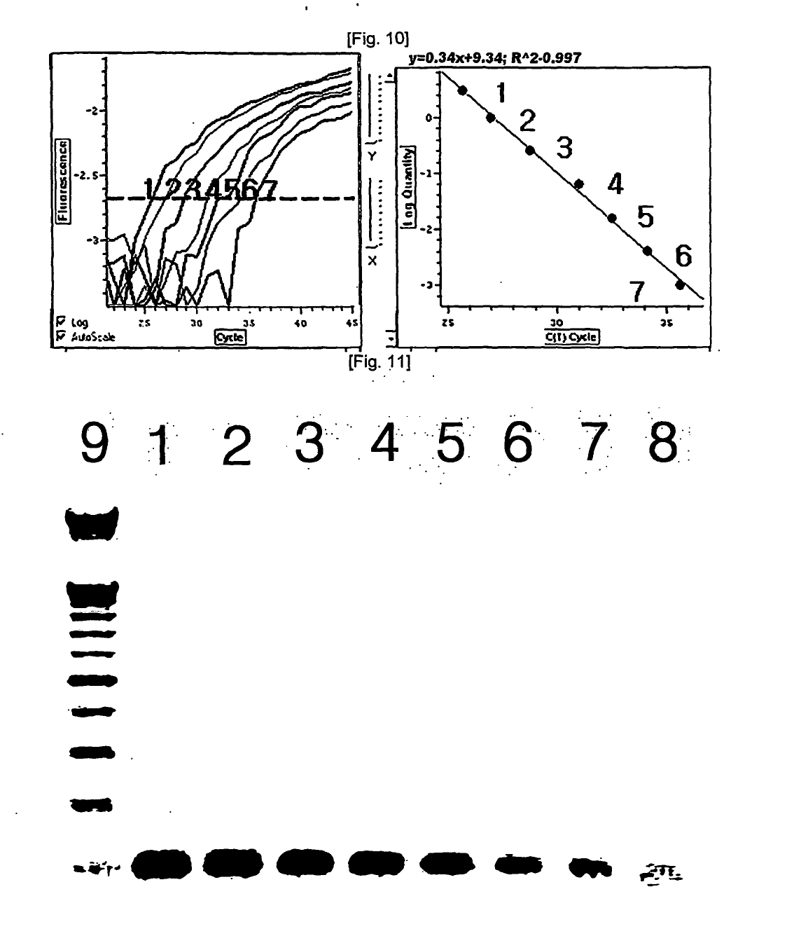

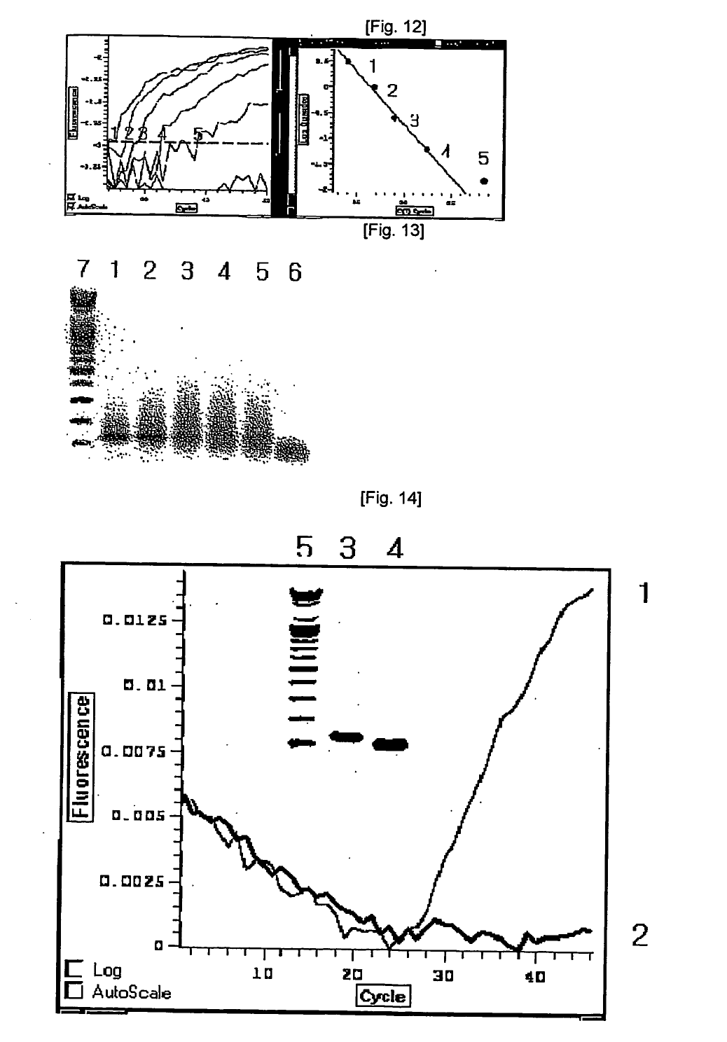

The X-axis of FIG. 12 represents a reaction cycle of PCR and the Y-axis represents measured fluorescence intensity. Peak 1 is the fluorescence intensity of 6 ng of tubercle DNA per a tube, the peak 2 is the fluorescence intensity of 2 ng of tubercle DNA per a tube, the peak 3 is the fluorescence intensity of 500 pg of tubercle DNA per a tube, the peak 4 is the fluorescence intensity of 125 pg of tubercle DNA per a tube, the peak 5 is the fluorescence intensity of 31.3 pg of tubercle DNA per a tube, the peak 6 is the fluorescence intensity of 7.8 pg of tubercle DNA per a tube and the peak 7 is the fluorescence intensity of 1.95 pg of tubercle DNA per a tube in PCR reaction.

FIG. 11 shows the result of a gel electrophoresis of serially diluted tubercle DNA. Lane 1 is the result of a gel electrophoresis of 6 ng of tubercle DNA per a tube, lane 2 is the result of a gel electrophoresis of 2 ng of tubercle DNA per a tube. Lane 3 is the result of a gel electrophoresis of 500 pg of tubercle DNA per a tube, lane 4 is the result of a gel electrophoresis of 125 pg of tubercle DNA per a tube and lane 5 is the result of a gel electrophoresis of 31.3 pg of tubercle DNA per a tube. Lane 6 is the result of a gel electrophoresis of 7.8 pg of tubercle DNA per a tube and lane 7 is the result of a gel electrophoresis of 1.95 pg of tubercle DNA per a tube in PCR reaction. Lane 8 is a result of a gel electrophoresis of a negative control and lane 9 represents 100 bp size marker.

### EXAMPLE 8

### Fixed Quantity PCR which Uses a Probe Labeled with DNA GREEN Phosphoramidite at the Middle Region

Fixed quantity real time PCR was carried out for tubercle DNA in order of concentration according to the reaction conditions in Example 5. Seq. ID No.1 and 2 produced in Example 2 were used as primers and at least one of oligonucleotides of Seq. ID No.13 to 17 was used as probes.

20 μl of reaction buffer was prepared in each tubes to perform PCR by using said each primer and primer.

The buffer was prepared by following procedures.

2 μl of 10× reaction buffer (200 mM Tris-HCl, 100 nm KCl, pH 9.0), 2 μl of 10 mM of dNTPs (2.5 mM dATP, 2.5 mM dGTP, 2.5 mM dCTP, 2.5 mM dTTP, respectively) and 20 mM of MgCl2 were added to the tubes to get the concentration of the mixture to be finally 1.4 mM. And Seq. ID Nos. 1 and 2, a primer for amplification of target nucleic acid, were added to get the final concentration to be 0.5 uM.

Seq. ID No. 16, a probe labeled with DNA GREEN phosphoramidite at the middle region, was added to the tubes to get the concentration to be 1.0 uM. And Clen Taq polymerase (BIONEER Co.) was added to each reaction tubes to become 0.12 U (unit). The tubes were filled with distilled water to get final volume to be 20 μl and spun down by micro centrifuge. Distilled water was used as a negative control.

Afterward, real-time PCR was carried out by using PCR machine (Opticon™, MJ Co.). In above PCR, pre-denaturing for 5 min. at 94° C., denaturing for 30 sec at 95° C., annealing for 50 sec at 56° C. and elongation for 40 sec at 72° C., were carried out for 46 times.

Fluorescence intensities of every annealing step were measured and an amplification curve of products of real-time PCR was established by using the measured data.

Afterward, the amplification curve was changed logarithmically and the threshold cycle[C (T)] was decided. Standard calibration curve was made and the linearity of fixed quantity PCR was decided.

PCR products were confirmed by performing a gel electrophoresis, which is prepared by a method of Example 4. Size of the amplified products obtained by using a primer labeled with DNA GREEN phosphoramidite at the middle region, was 127 bp.

As indicated in FIG. 12, left graph is the said log of amplification curve deciding the threshold cycle(C (T)). Right graph is the standard calibration curve of serially diluted tubercle DNA according to the threshold cycle[C (T)] curve.

Compared with the negative control, left graph shows an increase of fluorescence intensity as PCR reaction repeated. Right graph shows that the linearity (R_{—}2) is 0.993.

The X-axis of FIG. 12 represents a reaction cycle of PCR and the Y-axis represents measured fluorescence intensity. Peak 1 is a fluorescence intensity of 6 ng of tubercle DNA per a tube, the peak 2 is a fluorescence intensity of 2 ng of tubercle DNA per a tube, the peak 3 is a fluorescence intensity of 500 pg of tubercle DNA per a tube, the peak 4 is a fluorescence intensity of 125 pg of tubercle DNA per a tube and the peak 5 is a fluorescence intensity of 31.3 pg of tubercle DNA per a tube in PCR reaction.

FIG. 13 shows a result of a gel electrophoresis of serially diluted tubercle DNA. Lane 1 is a result of a gel electrophoresis of 6 ng of tubercle DNA per a tube, lane 2 is a result of a gel electrophoresis of 2 ng of tubercle DNA per a tube, lane 3 is a result of a gel electrophoresis of 500 pg of tubercle DNA per a tube, lane 4 is a result of a gel electrophoresis of 125 pg of tubercle DNA per a tube and lane 5 is a result of a gel electrophoresis of 31.3 pg of tubercle DNA per a tube in PCR reaction. Lane 6 is a result of a gel electrophoresis of a negative control and lane 7 represents 100 bp size marker.

### EXAMPLE 9

### PCR Cross Contamination Experiment which Uses a Probe Labeled with DNA GREEN Phosphoramidite at the 5′ End

Lambda DNA is separated and refined from heat inducible lysogen E. coli strain(dam-, dcm-) infected with lambda phage (Ci857 Sam7).

According to reaction condition of Example 4, a cross contamination reaction is performed by using a probe labeled with DNA GREEN phosphoramidite at the 5′ end.

At least one of oligonucleotides of Seq. ID No.8 to 12 was used as a probe in the present Example. Seq. ID No. 1 and 2 were used as primers for PCR of tubercle DNA and Seq. ID No. 6 and 7 were used as primers for PCR of lambda DNA.

20 μl of reaction buffer was prepared in each tubes to perform PCR by using said each primer and primer.

The buffer was prepared by following procedures.

For the PCR of tubercle DNA, 2 μl of 10× reaction buffer (200 mM Tris-HCl, 100 mM KCl, pH 9.0), 2 μl of 10 mM dNTPs (2.5 mM dATP, 2.5 mM dGTP, 2.5 mM dCTP, 2.5 mM dTTP, respectively) and 20 mM of MgCl2 were added to the tubes to get the final concentration of the mixture to be 2 mM respectively. And Seq. ID Nos. 1 and 2, primers for an amplification of target nucleic acid, were added to get the concentration to be 0.5 uM respectively.

Afterward, Seq. ID No. 10, a probe labeled with DNA GREEN phosphoramidite at the 5′ end, was added to the tubes to get the concentration to be 2.5 uM. And Clen Taq polymerase (BIONEER Co.) was added to each reaction tubes to become 0.2 U (unit). And 2 μl of refined tubercle DNA in Example 1 was added to each tube. The tubes were filled with distilled water to get final volume to be 20 μl and were spun down by micro centrifuge.

For the PCR of lambda DNA, 2 μl of 10× reaction buffer (200 mM Tris-HCl, 100 mM KCl, pH 9.0), 2 μl of 10 mM dNTPs (2.5 mM dATP, 2.5 mM dGTP, 2.5 mM dCTP, 2.5 mM dTTP, respectively) and 20 mM of MgCl2 were added to the tubes to get the concentration of the mixture to be finally 2 mM. And Seq. ID Nos. 6 and 7, a primer for amplification of target nucleic acid, were added to get the final concentration to be 0.5 uM.

Seq. ID No. 10, a probe labeled with DNA GREEN phosphoramidite at the 5′ end, was added to the tubes to get the concentration to be 2.5 uM. And Clen Taq polymerase (BIONEER Co.) was added to each reaction tubes to become 0.2 U. And 10 pg of lambda DNA was added to each tube. The tubes were filled with distilled water to get final volume to be 20 μl and spun down by micro centrifuge.

Afterward, real-time PCR was carried. In the PCR, pre-denaturing for 5 min. at 94° C., denaturing for 30 sec at 95° C., annealing for 60 sec at 55° C. and elongation for 40 sec at 72° C., were carried out for 44 times.

Fluorescence intensities of every annealing step were measured and an amplification curve of products of real-time PCR was established by using the measured data. PCR products were confirmed by performing a gel electrophoresis, which is prepared by a method of Example 4.

FIG. 14 shows the result of real time PCR of the tubercle DNA and the lambda DNA when Seq. ID No. 10 was added to both tubes. The fluorescence intensity of tubercle DNA was increased as reaction cycles were repeated but the fluorescence intensity of lambda DNA was not changed as reaction cycles were repeated. The X-axis of FIG. 14 represents PCR cycle and the Y-axis represents measured fluorescence intensity.

Lane 1 is a graph of the fluorescence intensity of each reaction cycle of tubercle DNA and Lane 2 is a graph of the fluorescence intensity of each reaction cycle of lambda DNA.

PCR products were confirmed by performing a gel electrophoresis, which is prepared by a method of Example 4. Size of the amplified products obtained by using a primer labeled with DNA GREEN phosphoramidite at the 5′ end, was 127 bp. Size of the amplified products of the lambda DNA was 100 bp.

In FIG. 14, Lane 3 is a result of a gel electrophoresis of real time PCR products of tubercle DNA and Lane 4 is a result of a gel electrophoresis of real time PCR products of lambda DNA. Lane 5 represents 100 bp size marker.

### EXAMPLE 10

### PCR Cross Contamination Experiment which Uses a Probe Labeled with DNA GREEN Phosphoramidite at the Middle Region

A cross contamination reaction of a probe labeled with phosphoramidite at the middle region was carried out according to the reaction condition of example 5.

At least one of Seq. ID No. 13 to 17 prepared in the present Example 2, was used as a probe. Seq. ID No. 1 and 2 were used as a primer for PCR of tubercle DNA and Seq. ID No. 6 and 7 were used as a primer for PCR of lambda DNA.

20 μl of reaction buffer was prepared in each tubes to perform PCR by using said each primer and primer.

The buffer was prepared by following procedures.

For the PCR of tubercle DNA, 2 μl of 10× reaction buffer (200 mM Tris-HCl, 100 mM KCl, pH 9.0), 2 μl of 10 mM dNTPs (2.5 mM dATP, 2.5 mM dGTP, 2.5 mM dCTP, 2.5 mM dTTP, respectively) and 20 mM of MgCl2 were added to the tubes to get the final concentration of the mixture to be 1.5 mM respectively. And Seq. ID Nos. 1 and 2, primers for an amplification of target nucleic acid, were added to get the concentration to be 0.5 uM.

Seq. ID No. 16, a probe labeled with DNA GREEN phosphoramidite at the middle region, was added to the tubes to get the concentration to be 1.0 uM. And Clen Taq polymerase (BIONEER Co.) was added to each reaction tubes to become 0.15 U (unit). And 1.5 μl of refined tubercle DNA in Example 1 was added to each tube. The tubes were filled with distilled water to get final volume to be 20 μl and were spun down by micro centrifuge.

For the PCR of lambda DNA, 2 μl of 10× reaction buffer (200 mM Tris-HCl, 100 mM KCl, pH 9.0), 2 μl of 10 mM of dNTPs (2.5 mM dATP, 2.5 mM dGTP, 2.5 mM dCTP, 2.5 mM dTTP, respectively) and 20 mM of MgCl2 were added to the tubes to get the concentration of the mixture to be finally 1.5 mM. And Seq. ID Nos. 6 and 7, a primer for amplification of target nucleic acid, were added to get the final concentration to be 0.5 uM.

Afterward, Seq. ID No. 16, a probe labeled with DNA GREEN phosphoramidite at the middle region, was added to the tubes to get the concentration to be 1.0 uM. And Clen Taq polymerase (BIONEER Co.) was added to each reaction tubes to become 0.15 U (unit). 10 pg of lambda DNA was added to each tube. The tubes were filled with distilled water to get final volume to be 20 μl and spun down by micro centrifuge.

Afterward, real-time PCR was carried out by using PCR machine (Opticon™, MJ Co.). In above PCR, pre-denaturing for 5 min. at 94° C., denaturing for 30 sec at 95° C., annealing for 50 sec at 56° C. and elongation for 40 sec at 72° C., were carried out for 46 times.

Fluorescence intensities of every annealing step were measured and an amplification curve of products of real-time PCR was established by using the measured data.

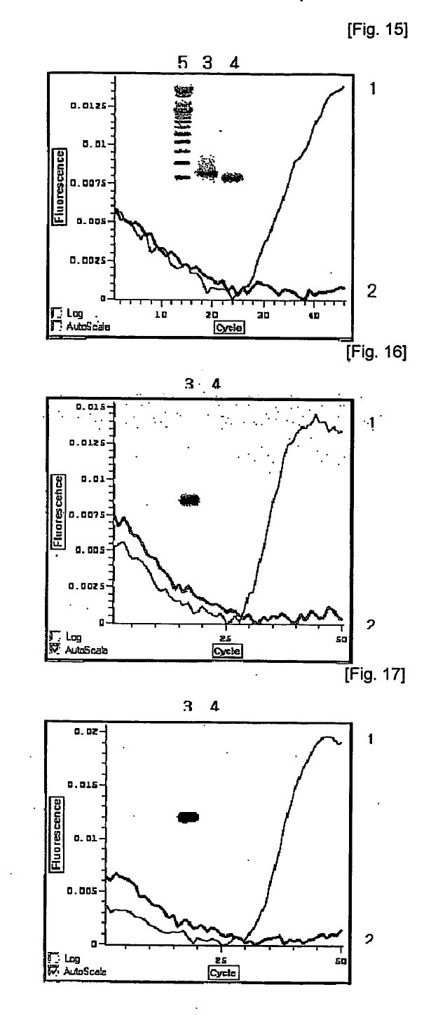

FIG. 15 shows the result of real-time PCR of the tubercle DNA and the lambda DNA when Seq. ID No. 16 was added to both tubes. The fluorescence intensity of tubercle DNA was increased as reaction cycles were repeated. However the fluorescence intensity of lambda DNA was not changed as reaction cycles were repeated. The X-axis of FIG. 15 represents PCR cycle and the Y-axis represents measured fluorescence intensity.

Lane 1 is a graph of the fluorescence intensity of each reaction cycle of tubercle DNA and Lane 2 is a graph of the fluorescence intensity of each reaction cycle of lambda DNA.

PCR products were confirmed by performing a gel electrophoresis, which is prepared by a method of Example 4. Size of the amplified products obtained by using a primer labeled with DNA GREEN phosphoramidite at the middle region, was 127 bp. Size of the amplified products of the lambda DNA was 100 bp.

In FIG. 15, Lane 3 is a result of a gel electrophoresis of real time PCR products of tubercle DNA and Lane 4 is a result of a gel electrophoresis of real time PCR products of lambda DNA. Lane 5 represents 100 bp size marker.

### EXAMPLE 11

### PCR, which Uses a Probe Labeled with DNA GREEN Phosphoramidite at the 3′ End of Base Sequence

VentR (exo-) DNA polymerase is a polymerase in which 3′→5′ proofreading exonuclease activity of VentR DNA polymerase is excluded. By using said polymerase, a probe labeled with a fluorescence dye at the 3′ end, can emit light with its specificity.

In the present Example, Seq. ID No. 19 and 20 in Table 1 and Table 2 were used as primers and Seq. ID No. 21 was used as a probe.

20 μl of reaction buffer was prepared in tubes to perform PCR by using said each primer and primer.

The buffer was prepared by following procedures.

2 μl of 10× reaction buffer (100 mM KCl, 10 mM (NH4)2SO4, 20 mM Tris-HCl(pH 8.8, @25° C.), 2 mM of MgSO4, 0.1% Trition X-100, NEB Co.), 3 μl of 10 mM of dNTPs (2.5 mM dATP, 2.5 mM dGTP, 2.5 mM dCTP, 2.5 mM dTTP, respectively) and 20 mM of MgSO4 were added into the tube to get each final concentration to be 2 mM, 3 mM and 4 mM respectively.

Afterward, Seq. ID Nos.19 and 20, primers for an amplification of a target nucleic acid, were added to the tubes to get the final concentration to be 0.6 uM respectively.

And then Seq. ID No. 21, a probe labeled with DNA GREEN phosphoramidite at the 3′ end, was added to the tubes to get the final concentration to be 1.0 uM and 2.5 uM respectively. And then VentR (exo-) DNA polymerase (NEB Co.) was added to each reaction tubes to become finally 0.25 U.

0.5 μl of refined tubercle DNA in Example 1 was added to each tube. And then the tubes were filled with distilled water to get each final volume to be 20 μl and were spun down by micro centrifuge.

After that, three step real-time PCR was carried out by using PCR machine (Opticon™, MJ Co.). In above PCR, pre-denaturing for 6 min. at 94° C., denaturing for 30 sec at 95° C., annealing for 60 sec at 53° C. and elongation for 50 sec at 72° C., were carried out for 50 times.

Fluorescence intensities of every annealing step were measured and an amplification curve of products of real-time PCR was established by using the measured data.

FIG. 16 indicates a result of real-time PCR in condition of 1.0 uM of Seq. ID No. 21 and 2 mM of MgCl2. The result showed an increase of fluorescence intensity as reaction cycles were repeated. In FIG. 16, the X-axis represents PCR cycles and the Y-axis represents measured fluorescence intensity.

Lane 1 is a graph of the fluorescence intensity measured in each PCR cycles by using tubercle DNA and 1.0 uM of probe labeled with DNA GREEN phosphoramidite at the 3′ end. Lane 2 is a graph of the fluorescence intensity measured in each PCR cycles by using distilled water and 1.0 uM of probe labeled with DNA GREEN phosphoramidite at the 3′ end.

PCR products were confirmed by performing a gel electrophoresis, which is prepared by a method of Example 4. Size of the amplified products obtained by using a primer labeled with DNA GREEN phosphoramidite at the 3′ end, was 118 bp.

As indicated in FIG. 16, Lane 3 is a result of the gel electrophoresis of PCR product of Lane 1, and Lane 4 is a result of the gel electrophoresis of PCR product of Lane 2.

### EXAMPLE 12

### PCR which Uses a Probe Labeled with DNA GREEN Phosphoramidite at the Both Ends (5′ End and 3′ End) of a Probe

VentR (exo-) DNA polymerase is a polymerase in which 3′→5′ proofreading exonuclease activity of VentR DNA polymerase is excluded. By using said polymerase, a probe labeled with a fluorescence dye at the 3′ end, can emit light with its specificity.

In the present Example, Seq. ID No.19 and 20 in Table 1 and Table 2 were used as primers and Seq. ID No. 22 was used as a probe.

20 μl of reaction buffer was prepared in tubes to perform PCR by using said each primer and primer.

The said buffer was prepared by following procedures.

2 μl of 10× reaction buffer (100 mM KCl, 10 mM (NH4)2SO4, 20 mM Tris-HCl (pH 8.8, @25° C.), 2 mM of MgSO4, 0.1% Trition X-100, NEB Co.), 3 μl of 10 mM of dNTPs (2.5 mM dATP, 2.5 mM dGTP, 2.5 mM dCTP, 2.5 mM dTTP, respectively) and 20 mM of MgSO4 were added into the tube to get each final concentration to be 2 mM, 3 mM and 4 mM respectively.

Afterward, Seq. ID Nos.19 and 20, primers for an amplification of a target nucleic acid, were added to the tubes to get the final concentration to be 0.6 uM respectively.

And then Seq. ID No. 22, a probe labeled with DNA GREEN phosphoramidite at both 3′ and 5′ end, was added to the tubes to get the final concentration to be 1.0 uM and 2.5 uM respectively. And then VentR (exo-) DNA polymerase (NEB Co.) was added to each reaction tubes to become finally 0.25 U.

0.5 μl of refined tubercle DNA in Example 1 was added to each tube. And then the tubes were filled with distilled water to get each final volume to be 20 μl and were spun down by micro centrifuge.

After that, three step real-time PCR was carried out by using PCR machine (Opticon™, MJ Co.). In above PCR, pre-denaturing for 6 min. at 94° C., denaturing for 30 sec at 95° C., annealing for 60 sec at 53° C. and elongation for 50 sec at 72° C., were carried out for 50 times.

Fluorescence intensities of every annealing step were measured and an amplification curve of products of real-time PCR was established by using the measured data.

FIG. 17 indicates a result of real-time PCR in condition of 1.0 uM of Seq. ID No. 22 and 2 mM of MgCl2. The result showed an increase of fluorescence intensity as reaction cycles were repeated. In FIG. 17, the X-axis represents PCR cycles and the Y-axis represents measured fluorescence intensity.

Lane 1 is a graph of the fluorescence intensity measured in each PCR cycles by using tubercle DNA and 1.0 uM of probe labeled with DNA GREEN phosphoramidite at both 3′ and 5′ end. Lane 2 is a graph of the fluorescence intensity measured in each PCR cycles by using distilled water and 1.0 uM of probe labeled with DNA GREEN phosphoramidite at both 3′ end and 5′ end.

PCR products were confirmed by performing a gel electrophoresis, which is prepared by a method of Example 4. Size of the amplified products obtained by using a primer labeled with DNA GREEN phosphoramidite at both 3′ end and 5′ end, was 118 bp.

As indicated in FIG. 17, Lane 3 is a result of the gel electrophoresis of PCR product of Lane 1, and Lane 4 is a result of the gel electrophoresis of PCR product of Lane 2.

1. A fluorescent probe for real-time detection of amplification of nucleic acid, wherein a fluorescent dye of which intensity of fluorescence is varied when the dye is intercalated into a double-stranded nucleic acid, is connected with an oligonucleotide of which base sequence is complementary with at least a part of said nucleic acid.

2. The fluorescent probe according to claim 1, wherein said fluorescent dye is connected with at least one selected from the 5′ end region, the 3′ end region, and the middle region of oligonucleotide.

3. The fluorescent probe according to claim 1, wherein the 3′ end of said fluorescent probe is blocked so that replication cannot begin from said 3′ end.

4. The fluorescent probe according to claim 1, wherein said fluorescent dye is selected from the group of Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide(Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide(PI) and derivatives thereof and Cy-dyes derivatives.

5. The fluorescent probe according to claim 1, wherein the fluorescent dye is labeled at said oligonucleotide by being bonded with or being replaced with a base of said oligonucleotide.

6. The fluorescent probe according to claim 1, wherein said fluorescent probe is hybridized with at least a portion of region which is inside the range of one (1) to fifteen (15) bases from the base on which 3′ end of said primer is combined.

7. The fluorescent probe according to claim 1, wherein the oligonucleotide is composed of ten (10) to forty (40) bases.

8. The fluorescent probe according to claim 1, wherein said oligonucleotide comprises the base sequence selected from the group which consists of the Seq. ID Nos. 1 through 22.

9. A real-time detection method of nucleic acid amplification, comprising the steps of:
i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase;
ii) denaturing said double-stranded nucleic acid into single strands by heating the aqueous buffer prepared in step i) up to 93° C. to 96° C.;
iii) annealing said pair of primers with said single strand by cooling the solution obtained in step ii) up to 50° C. to 57° C.;
iv) replicating said single-stranded nucleic acid by heating the solution obtained from step iii) up to 70° C. to 74° C.;
v) denaturing said replicated nucleic acid into single strands by heating the solution obtained in step iv) up to 93° C. to 96° C.;
vi) annealing said fluorescent probe with said single-stranded nucleic acid by cooling the solution obtained in step v) up to 50° C. to 57° C.;
vii) measuring an intensity of the fluorescence emitted from the solution obtained in step vi); and
vi) repeating more than one steps iv) through vii).

10. The real-time detection methods according to claim 9, wherein said step vii) is preformed concurrently with the step vi).

11. A detection method of initial amount of a nucleic acid in a sample, comprising the steps of:
i) producing an aqueous buffer which contains a nucleic acid, a pair of primers for amplification of said nucleic acid, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides and DNA polymerase;
ii) denaturing said double-stranded nucleic acid into single strands by heating the aqueous buffer prepared in step i) up to 93° C. to 96° C.;
iii) annealing said pair of primers with said single strand by cooling the solution obtained in step ii) up to 50° C. to 57° C.;
iv) replicating said single-stranded nucleic acid by heating the solution obtained from step iii) up to 70° C. to 74° C.;
v) denaturing said replicated nucleic acid into single strands by heating the solution obtained in step iv) up to 93° C. to 96° C.;
vi) annealing said fluorescent probe with said single-stranded nucleic acid by cooling the solution obtained in step v ) up to 50° C. to 57° C.;
vii) measuring an intensity of the fluorescence emitted from the solution obtained in step vi);
viii) repeating more than one steps iv) through vii);
ix) establishing a standard calibration curve which indicates the correlation between the log value of an initial amount of the nucleic acid and a threshold cycle shown by the performance of above steps i) through viii), by using a sample of which an initial amount of the nucleic acid is known; and
x) detecting an initial amount of the nucleic acid based on the log value which corresponds to the threshold cycle obtained from the performance of said steps i) through viii), by referring to the standard calibration curve obtained in step ix).

12. The detection method according to claim 11, wherein said step vii) is preformed concurrently with the step vi).

13. A composition for the amplification of a nucleic acid which comprises i) a pair of primers for amplification of said nucleic acid; ii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iii) DNA polymerase; and iv) four (4) kinds of nucleotides.

14. The composition according to claim 13, wherein said fluorescent dye is connected with at least one selected from the 5′ end region, the 3′ end region, and the middle region of oligonucleotide.

15. The composition according to claim 13, wherein the 3′ end of said fluorescent probe is blocked so that replication cannot begin from said 3′ end.

16. The composition according to claim 13, wherein said fluorescent dye is selected from the group of Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide(Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide(PI) and derivatives thereof and Cy-dyes derivatives.

17. The composition according to claim 13, wherein the fluorescent dye is labeled at said oligonucleotide by being bonded with or being replaced with a base of said oligonucleotide.

18. The composition according to claim 13, wherein said fluorescent probe is hybridized with at least a portion of region which is inside the range of one (1) to fifteen (15) bases from the base on which 3′ end of said primer is combined.

19. The composition according to claim 13, wherein the oligonucleotide is composed of ten (10) to forty (40) bases.

20. The composition according to claim 13, wherein said oligonucleotide comprises the base sequence selected from the group which consists of the Seq. ID Nos. 1 through 22.

21. The composition according to claim 13, wherein said composition is dried in vacuum.

22. A real-time detection method of the nucleic acid amplification, comprising the steps of:
i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase;
ii) replicating a single-stranded cDNA by heating the aqueous buffer prepared in step i) up to 42° C. to 50° C.;
iii) denaturing a primer for a reverse transcription and a reverse transcriptase from said single-stranded cDNA by heating the solution obtained from said step ii) up to 93° C. to 96° C.;
iv) annealing the pair of primers with said single-stranded nucleic acid by cooling the solution obtained from said step iii) up to 50° C. to 57° C.;
v) replicating said single-stranded nucleic acid by heating the solution obtained from step iv) up to 70° C. to 74° C.;
vi) denaturing said replicated nucleic acid into single strands by heating the solution obtained from step v) up to 93° C. to 96° C.;
vii) annealing said fluorescent probe with said single-strand nucleic acid by cooling the solution obtained from step vi) up to 50-57° C.;
viii) measuring an intensity of the fluorescence emitted from the solution obtained in step vii); and
ix) repeating more than one steps v) through viii).

23. The detection method according to claim 22, wherein said step viii) is preformed concurrently with the step vii).

24. A detection method of the initial amount of a nucleic acid in a sample, comprising the steps of:
i) producing an aqueous buffer which contains a nucleic acid, a pair of primers for amplification of said nucleic acid, a primer for reverse transcription, a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid, four (4) kinds of nucleotides, DNA polymerase and reverse transcriptase;
ii) replicating a single-stranded cDNA by heating the aqueous buffer prepared in step i) up to 42° C. to 50° C.;
iii) denaturing a primer for a reverse transcription and a reverse transcriptase from said single-stranded cDNA by heating the solution obtained from said step ii) up to 93° C. to 96° C.;
iv) annealing the pair of primers with said single-stranded nucleic acid by cooling the solution obtained from said step iii) up to 50° C. to 57° C.;
v) replicating said single-stranded nucleic acid by heating the solution obtained from step iv) up to 70° C. to 74° C.;
vi) denaturing said replicated nucleic acid into single strands by heating the solution obtained from step v) up to 93° C. to 96° C.;
vii) annealing said fluorescent probe with said single-strand nucleic acid by cooling the solution obtained from step vi) up to 50-57° C.;
viii) measuring an intensity of the fluorescence emitted from the solution obtained in step vii);
ix) repeating more than one steps v) through viii);
x) establishing a standard calibration curve which indicates the correlation between the log value of an initial amount of the nucleic acid and a threshold cycle shown by the performance of above steps i) through ix), by using a sample of which an initial amount of the nucleic acid is known; and
xi) detecting an initial amount of the nucleic acid based on the log value which corresponds to the threshold cycle obtained from the performance of said steps i) through ix), by referring to the standard calibration curve obtained in step ix).

25. The detection method according to claim 24, wherein said step viii) is preformed concurrently with the step vii).

26. A composition for the amplification of a nucleic acid which comprises i) a pair of primers for amplification of said nucleic acid; ii) a primer for reverse transcription iii) a fluorescent probe wherein a fluorescent dye of which intensity of fluorescence is varied when the dye is intercalated into a double-stranded nucleic acid, is connected with an oligonucleotide of which base sequence is complementary with at least a part of said nucleic acid; iv) DNA polymerase; v) a reverse transcriptase and iv) four (4) kinds of nucleotides.

27. The composition according to claim 26, wherein said fluorescent dye is connected with at least one selected from the 5′ end region, the 3′ end region, and the middle region of oligonucleotide.

28. The composition according to claim 26, wherein the 3′ end of said fluorescent probe is blocked so that replication cannot begin from said 3′ end.

29. The composition according to claim 26, wherein said fluorescent dye is selected from the group of Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide(Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide(PI) and derivatives thereof and Cy-dyes derivatives.

30. The composition according to claim 26, wherein the fluorescent dye is labeled at said oligonucleotide by being bonded with or being replaced with a base of said oligonucleotide.

31. The composition according to claim 26, wherein said fluorescent probe is hybridized with at least a portion of region which is inside the range of one (1) to fifteen (15) bases from the base on which 3′ end 5′ endid primer is combined.

32. The composition according to claim 26, wherein the oligonucleotide is composed of ten (10) to forty (40) bases.

33. The composition according to claim 26, wherein said oligonucleotide comprises the base sequence selected from the group which consists of the Seq. ID Nos. 1 through 22.

34. The composition according to claim 26, wherein said composition is dried in vacuum.
